(19) United States
(12) Patent Application Publication
(10) Pub. No.: US 2023/0243814 A1
(43) Pub. Date: Aug. 3, 2023

(54) CHEMICAL CLASSIFICATION SYSTEM AND METHOD FOR PLANTS

(71) Applicant: STEEP HILL, INC., Berkeley, CA (US)

(72) Inventors: Thomas BLANK, Palmetto, FL (US); Reggie GAUDINO, San Marcos, CA (US)

(21) Appl. No.: 18/011,072

(22) PCT Filed: Jun. 17, 2021
(86) PCT No.: PCT/US2021/037896

§ 371 Date: Dec. 16, 2022

Related U.S. Application Data
(60) Provisional application No. 63/040,708, filed on Jun. 18, 2020.

(51) Int. Cl. G01N 33/50

(52) U.S. Cl. CPC G01N 33/5097

(57) ABSTRACT

This technology relates in part to methods of classifying plant strains, such as Cannabis plant strains, in a manner that clusters them into clades based on shared terpene profiles. The methods provided herein permit plant strains with desired characteristics/phenotypes to be identified for use in various applications, such as agriculture (e.g., selecting strains for breeding desired characteristics) and medicine (e.g., therapeutic activity).

### CROSS-REFERENCE TO RELATED APPLICATIONS

This application claims priority to U.S. Provisional Patent Application No. 63/040,708, filed on Jun. 18, 2020, entitled CHEMICAL CLASSIFICATION SYSTEM AND METHOD FOR PLANTS, naming Thomas Blank et al. as inventors, and designated by attorney docket number SHL-1003-PV, the entire content of which is incorporated herein by reference for all purposes.

### FIELD

The technology relates in part to a method of classifying plant cultivars into clades, based on their terpene content, and to methods of using plant cultivars based on such classification. The clades can be used to identify plant cultivars of a desired phenotype for methods of agricultural, medicinal or industrial use.

### BACKGROUND

The classification of plant cultivars in a manner that permits easy selection of a plant for a desired application, such as in agriculture (e.g., for breeding to obtain desired phenotypes) or medicine (to obtain desired therapeutic effects) can be challenging. This particularly is the case when the cultivars cannot readily be delineated by genotype due to decades or even centuries of changes that occur from factors such as random human selection, inbreeding and cross breeding, natural outcrossing and genome mixing.

For example, historically and to this day, Cannabis plants are broadly classified as being an Indica strain, a Sativa strain, or a Hybrid strain, i.e., having both Indica and Sativa lineage. It is thought that Indica strains are physically sedating, Sativa strains provide energizing cerebral effects and Hybrids provide a balance of Indica and Sativa effects. The classification, however, is in fact primarily morphological: Sativa strains have a lighter colored, pointy shaped leaf and a taller plant, while the species identified as Indica are a shorter plant with broader, dark colored leaves. It has been found that several so-called “Indica” strains can produce energizing effects, and several so-called “Sativa” strains can produce sedating effects. In addition, decades of crossbreeding have left few, if any, pure Indicas or Sativas. Large genetic variance, differences in phenotypes and differences in chemical profiles have been observed within even identically named strains, making classifying strains or cultivars according to genotype, phenotype or chemical profiles a challenge.

Due to problems, such as those noted above, in reliably identifying plant cultivars, a method is needed for classifying plant cultivars in a manner that permits the selection of phenotypes according to their intended use, e.g., for breeding or for therapeutic use.

### SUMMARY

Provided herein are methods of classifying a plurality of cultivars or strains of a plant according to chemotype, wherein the methods include:
(a) obtaining a plant sample from each of the plurality of strains;
(b) for each plant sample, obtaining a measured amount of one or more individual analytes in the sample, and a measured amount of the total analytes in the sample, wherein the analytes belong to the same chemical class;
(c) for each plant sample, based on the measured amounts in (b):
(i) determining the abundance of the one or more individual analytes in the sample relative to the total amount of analytes in the sample, thereby obtaining the relative abundance of the one or more individual analytes in the sample,
(ii) determining the order of relative abundance, from highest to lowest relative abundance or from lowest to highest relative abundance, of the one or more individual analytes in the sample, and
(iii) based on (i) and (ii), determining an abundance profile of the analytes for each plant sample;
(d) optionally, for each plant sample, determining whether the sample is an outlier and, if the plant sample is an outlier, not subjecting the sample to (e) and (f) or,
determining the difference between the original analyte abundance profile of the sample and the analyte abundance profile that renders the sample an outlier and, based on the difference, reconstructing the original analyte profile of the sample before subjecting the sample to (e) and (f);
(e) for each plant sample not identified as an outlier, normalizing the measured amounts of the one or more individual analytes, thereby obtaining, for each plant sample, a normalized abundance profile that includes normalized analyte levels of the one or more individual analytes; and
(f) based on the normalized abundance profiles of the analytes for each plant sample, assigning plant samples containing the same normalized abundance profiles to a group, wherein each group is a primary clade that comprises plant samples of the same chemotype.

The term “strain” is used interchangeably herein with “cultivar” (cultivated variety) or “variety” and refers to a species of a family of plants, such as a species of a Cannabis plant. A cultivar generally has been cultivated for desirable characteristics, such as color, shape, smell, medicinal use, etc., that are maintained during propagation. Phrases such as “plurality of strains of a plant” or equivalent phrases, as used herein, refers to multiple species of the same plant, e.g., a variety of strains or cultivars of Cannabis.

In certain embodiments, the methods can further include identifying one or more secondary clades in at least one primary clade:
(1) for each plant sample in at least one primary clade, the identity and/or normalized measured amount of (i) one or more additional analytes, or (ii) a mixture of one or more individual analytes in (a) and one or more additional analytes is obtained, where the additional analytes are associated with heredity and/or a known therapeutic effect and where the additional analytes are different than the individual analytes analyzed to obtain primary clades;
(2) for each plant sample, based on the identity and/or normalized measured amount of amount of (i) or (ii), obtaining one or more profiles selected from among a heredity profile of analytes and a therapeutic profile of the analytes of (i) or (ii); and
(3) identifying plant samples within each primary clade that contain the same heredity profiles and/or therapeutic profiles, as belonging to the same secondary clade.

In certain embodiments, the plant sample is identified as an outlier if the total amount of the analyte in the sample is less than a threshold amount, or, when comparing the measured amount of at least one individual first analyte to a reference amount of the first analyte, and/or comparing the ratio of the measured amounts of at least one individual first analyte and at least one individual second analyte to a reference ratio of the amounts of the first analyte and the second analyte, if the measured amount and/or ratio is different than the reference amount or ratio, the plant sample can be identified as an outlier.

In certain embodiments of the methods provided herein, plant samples are identified as containing the same abundance profiles or normalized abundance profiles by performing a clustering analysis to obtain one or more clusters, where each cluster is assigned an average abundance profile. The average abundance profile can be represented as a centroid vector, the abundance profile or normalized abundance profile of each plant sample can be represented as a vector, and plant samples whose normalized abundance profile vector distances to the centroid vector are at or below a minimum value are identified as having the same abundance profiles and belonging to the same cluster. Each cluster that contains a unique centroid vector that is different than the centroid vectors of all the other clusters obtained by the clustering analysis is identified as a primary clade.

In embodiments of the methods provided herein, plant samples are identified as containing the same heredity profiles or therapeutic profiles in the secondary clades by performing a clustering analysis to obtain one or more clusters, where each cluster is assigned an average heredity profile or an average therapeutic profile, each average heredity profile or the average therapeutic profile is represented as a centroid vector, each heredity profile or therapeutic profile of each plant sample is represented as a vector, and plant samples whose heredity profile vector or therapeutic profile vector distances to the centroid vector are at or below a minimum value are identified as having the same heredity profiles or therapeutic profiles and belonging to the same cluster. Each cluster containing a unique centroid vector that is different than the centroid vectors of all the other clusters obtained by the clustering analysis is identified as a secondary clade.

In any of the methods provided herein, if the primary analytes used to construct primary clades are also used to construct secondary clades, the primary analytes can be modified by a weighting factor to account for the abundancy, which often can be orders of magnitude larger than the secondary analytes used to construct the secondary clades. For example, if the secondary clade is constructed based on plant strains containing the same therapeutic profile, the weighting factor for the primary analytes can be based on potency.

In certain embodiments, a subset of the analytes of the plant strains are analyzed for classification into primary clades according to the methods provided herein. In embodiments, the subset includes individual analytes that represent 3%, 4%, 5%, 6%, 7%, 8%, 9%, 10%, 11%, 12%, 13%, 14%, 15%, 16%, 17%, 18%, 19%, 20% or more by weight of the total amount by weight of all the analytes recovered from each plant strain.

In certain embodiments of the methods provided herein, the analytes are terpenes.

In embodiments of the methods provided herein, the plant strains are Cannabis strains. In certain embodiments, the terpenes of the Cannabis plant strains that are analyzed to obtain abundance profiles of the plant strains include beta myrcene, beta caryophyllene, limonene, alpha pinene, beta farnesene, and terpinolene. In embodiments, the terpenes of the Cannabis plant strains that are analyzed to obtain abundance profiles of the plant strains include terpenes that are co-products of beta myrcene, beta caryophyllene, limonene, alpha pinene, beta farnesene, and/or terpinolene, such as, for example, humulene, beta pinene, and alpha farnesene.

In embodiments, when the plant strains are Cannabis strains, determining whether a sample from a plant strain is an outlier for exclusion from analysis or for adjustment prior to analysis according to the methods provided herein can include measuring the ratio of tetrahydrocannabinol (THC) to tetraydrocannabinolic acid (THCA) and, if the ratio is at or above a threshold value, identifying the sample as an outlier. In certain embodiments, if the ratio is at or above 1:10, i.e., 10% or more of the THCA is decarboxylated to produce THC (e.g., due to processing, storage, etc. of the plant samples), the plant sample is identified as an outlier. In embodiments of the methods provided herein, determining whether the sample is an outlier can include one or more of:
1) if the ratio of beta caryophyllene:humulene is not between 2:1 to 6:1, identifying the sample as an outlier;
2) if the amount of alpha pinene is greater than two times the limit of quantitation (LOQ), beta pinene must be detected or the sample is identified as an outlier;
3) if beta pinene is at limit of quantitation (LOQ), alpha pinene must be detected or the sample is identified as an outlier;
4) if the ratio of alpha pinene:beta pinene is not between 0.3:1 to 6:1, identifying the sample as an outlier;
5) if the ratio of terpinolene:3-carene is not between 10:1 to 38:1, identifying the sample as an outlier;
6) if the ratio of terpinolene:alpha phellandrene is not between 5:1 to 30:1, identifying the sample as an outlier;
7) if the ratio of terpinolene:alpha pinene is not between 20:1 to 100:1, identifying the sample as an outlier;
8) if the ratio of alpha terpineol:fenchol is not between 0.3:1 to 2.5:1, identifying the sample as an outlier;
9) if the ratio of terpinolene:gamma terpinene ratios is not between 20:1 to 120:1, identifying the sample as an outlier;
10) if the sample comprises about or less than about 0.7, 0.75, 0.8, 0.85, 0.9, 0.95 or 1% total terpenes by weight, based on the total dry weight of the sample, identifying the sample as an outlier; and
11) if the THC content of the sample is 10% or more of the THCA content, identifying the sample as an outlier.

In embodiments of the methods provided herein, if the sample contains about or less than about 0.9% total terpenes by weight, based on the total dry weight of the sample, the sample is identified as an outlier. The outlier sample can be excluded from analysis according to the methods provided herein, or the difference can be determined between the original analyte (e.g., terpene) abundance profile of the sample and the abundance profile that renders the sample an outlier and, based on the difference, the original analyte profile of the sample can be reconstructed before subjecting the sample to further analysis to construct primary and/or secondary clades. Determining the difference between the original terpene abundance profile of the sample and the terpene abundance profile that renders the sample an outlier can include, in embodiments, determining the decay profile of one or more terpenes in the sample, determining the storage time of the sample, identifying and/or quantitating terpene degradation products in the sample and/or determining the estimated dissipation of one or more terpenes in the sample.

In certain embodiments of the methods provided herein, one or more analytes used to obtain heredity and/or therapeutic profiles to identify secondary clades has a low volatilization rate. In embodiments, the one or more analytes is/are terpene(s). In certain embodiments, the one or more terpenes are selected from among monoterpene alcohols, sesquiterpenes, sesquiterpene alcohols or combinations thereof. In embodiments, the one or more terpenes are selected from among alpha bisabolol, alpha terpineol, guiaol, nerolidol, fenchol and linalool.

In certain embodiments of the methods provided herein, at least one secondary clade is obtained based on scoring one or more of the analytes for heredity, thereby obtaining at least one secondary clade wherein the plant strains that are members of the clade share the same average heredity profile. In embodiments, the analytes are terpenes. In certain embodiments, the terpenes that are scored for heredity include one or more terpenes selected from among monoterpene alcohols, sesquiterpenes, sesquiterpene alcohols or combinations thereof. In embodiments, the terpenes that are scored for heredity include one or more terpenes selected from among alpha bisabolol, alpha terpineol, guiaol, nerolidol, fenchol and linalool. In certain embodiments, the average heredity profile can further be correlated with therapeutic activity, thereby obtaining an average therapeutic profile for the secondary clade.

In embodiments of the methods provided herein, at least one secondary clade is obtained based on scoring one or more of the analytes for one or more therapeutic effects, thereby obtaining at least one secondary clade wherein the plant strains that are members of the clade share the same average therapeutic profile. In embodiments, the analytes are terpenes. In certain embodiments, at least one secondary clade is obtained based on scoring one or more of the terpenes for one or more therapeutic effects, thereby obtaining at least one secondary clade wherein the plant strains that are members of the clade share the same average therapeutic profile. In certain embodiments, the therapeutic effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects. In embodiments, at least one therapeutic effect is AChEI and in certain embodiments, the analytes are terpenes and the terpenes that are scored include one or more terpenes selected from among alpha pinene, eucalyptol, 3 carene, alpha terpinene, gamma terpinene, cis ocimene, trans ocimene and beta caryophyllene oxide. In certain embodiments, at least one therapeutic effect is analgesic and in embodiments, the analytes are terpenes and the terpenes that are scored comprise one or more terpenes selected from among alpha bisabolol, alpha terpineol, alpha phellandrene and nerolidol.

In certain embodiments of the methods provided herein, when at least one secondary clade is obtained based on scoring one or more of the analytes for one or more therapeutic effects, the therapeutic effect is on or through the brain waves. In embodiments, the therapeutic effect on or through the brain waves is gender selective. In embodiments, the terpenes that are scored for their therapeutic effect on brain waves include one or more terpenes selected from terpinolene, (+) limonene, (+) alpha pinene and (+) beta pinene.

In embodiments of the methods provided herein, the number of individual analytes whose amounts are measured in the plant strain samples to obtain abundance profiles of the plant strains can be between about 5 individual analytes to about 45, 50, 55, 60, 65, 70, 75, 80, 85, 90, 95, 100 or more individual analytes. In certain embodiments, the analytes are terpenes. In embodiments, the number of terpenes whose amounts are measured in the plant strain samples to obtain abundance profiles of the plant strains can be between about 10 terpenes to about 45, 50, 55, 60, 65, 70, 75, 80, 85, 90, 95, 100 or more terpenes and in embodiments, the number of terpenes whose amounts are measured in the plant strain samples to obtain abundance profiles of the plant strains can be between about 20 terpenes to about 45, 50, 55, 60, 65 or 70 terpenes. In certain embodiments, the number of terpenes whose amounts are measured in the plant strain samples to obtain abundance profiles of the plant strains is 43. In certain embodiments, the number of terpenes analyzed to obtain abundance, heredity, therapeutic or other profiles to classify the plant strains into clades is a subset of the number of terpenes whose amounts are measured in the plant strain samples. In embodiments, the number of terpenes in the subset is 10, 11, 12, 13, 14, 15, 16, 17, 18, 19 or 20 or more terpenes. In certain embodiments, the number of terpenes in the subset is 20 and in embodiments, the number of terpenes in the subset is 17.

In certain embodiments of the methods provided herein, the analytes are terpenes and the terpenes include one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol. Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In certain embodiments of the methods provided herein, when the analytes are terpenes, at least one of the terpenes analyzed to obtain abundance profiles for the library of plant strains used to construct primary clades is beta farnesene.

In embodiments of the methods provided herein, the number of terpenes analyzed to obtain abundance profiles for the library of plant strains used to construct primary clades is at least 3, 4, 5, 6, 7, 8, 9, 10, 11 or 12 terpenes. In certain embodiments, the number of terpenes analyzed to obtain abundance profiles for the library of plant strains used to construct primary clades is at least 6 terpenes, or 6 terpenes. In embodiments, the 6 terpenes are beta myrcene, beta caryophyllene, limonene, alpha pinene, beta farnesene and terpinolene. In embodiments, the number of terpenes analyzed to obtain abundance profiles for the library of plant strains used to construct primary clades is at least 9 terpenes, or 9 terpenes. In certain embodiments, the 9 terpenes are beta myrcene, beta caryophyllene, limonene, alpha pinene, beta farnesene, terpinolene, humulene, beta pinene, alpha farnesene.

In certain embodiments, the methods provided herein include obtaining a classification system based on the primary and/or secondary clades that are identified. In embodiments, the classification system can include one or more primary clades and in certain embodiments, the classification system can include one or more primary clades and one or more secondary clades. In certain embodiments, the number of primary clades is 3, 4, 5, 6, 7, 8, 9, 10, 11 or 12 and, in embodiments, the number of primary clades is 7.

Also provided herein is a classification system obtained by the methods provide herein. The classification systems provided herein can include:
(a) a first classification tier containing one or more primary clades, where the one or more primary clades all contain one or more strains of plants belonging to the same genus and where each primary clade contains one or more strains of plants belonging to the same genus that share a unique abundance profile of analytes that is different than the abundance profiles of analytes of the strains of plants in the other primary clades; and
(b) a second classification tier, containing one or more secondary clades, where:
the plant strains or a subset thereof in at least one primary clade are grouped into one or more secondary clades, where each secondary clade contains one or more strains of plants that share at least one unique profile selected from among (i) a unique heredity profile of analytes, and/or (iii) a unique therapeutic profile of analytes, where the shared unique profile/profiles of the plants in each secondary clade are different than the corresponding profiles of the plants in the other secondary clades,
the profiles in the second classification tier contain analytes that are different than the analytes of the profiles in the first classification tier, or the profiles in the second classification tier contain analytes that are a mixture of one or more analytes of the profiles in the first classification tier and one or more analytes that are different than the analytes of the profiles in the first classification tier, and
the analytes in the first classification tier and the analytes in the second classification tier belong to the same chemical class.

In certain embodiments of the classification systems provided herein, the analytes are terpenes and in embodiments, the plant strains are Cannabis strains. In certain embodiments, the terpenes include one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol. β-eudesmol. Hedycaryol, γ-eudesmol. Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In certain embodiments of the systems provided herein, the abundance profiles are obtained based on the abundances of at least 5, 6, 7, 8, 9, 10, 11 or 12 terpenes in each plant strain. In embodiments, the abundance profiles are obtained based on the abundances of at least 6 terpenes and in certain embodiments, the abundance profiles are obtained based on the abundances of 6 terpenes. In embodiments, the 6 terpenes are beta myrcene, beta caryophyllene, limonene, alpha pinene, beta farnesene and terpinolene in embodiments, the abundance profiles are obtained based on the abundances of at least 9 terpenes and in certain embodiments, the abundance profiles are obtained based on the abundances of 9 terpenes. In embodiments, the 9 terpenes are beta myrcene, beta caryophyllene, limonene, alpha pinene, beta farnesene, terpinolene, humulene, beta pinene and alpha farnesene.

In certain embodiments of the systems provided herein, the analytes are terpenes and the systems provided herein include primary clades based on abundance profiles where at least one of the terpenes is beta farnesene.

In certain embodiments of the systems provided herein, the analytes are terpenes and the total number of abundance, heredity and/or therapeutic profiles are obtained based on the abundance, heredity scoring and/or therapeutic scoring of 10, 11, 12, 13, 14, 15, 16, 17, 18, 19 or 20 or more terpenes. In embodiments, the total number of abundance, heredity and/or therapeutic profiles are obtained based on the abundance, heredity scoring and/or therapeutic scoring of 20 terpenes and in certain embodiments, the total number of abundance, heredity and/or therapeutic profiles are obtained based on the abundance, heredity scoring and/or therapeutic scoring of 17 terpenes.

In any of the systems provided herein, in certain embodiments, when the analytes are terpenes, at least one secondary clade is obtained based on scoring one or more of the terpenes for heredity, where the plant strains that are members of the clade share the same average heredity profile. In embodiments, the terpenes that are scored for heredity include one or more terpenes selected from among monoterpene alcohols, sesquiterpenes, sesquiterpene alcohols or combinations thereof. In certain embodiments, the terpenes that are scored for heredity include one or more terpenes selected from among alpha bisabolol, alpha terpineol, guiaol, nerolidol, fenchol and linalool. In embodiments, the average heredity profile can further be correlated with therapeutic activity and the secondary clade can contain an average heredity profile and an average therapeutic profile.

In any of the systems provided herein, in certain embodiments, when the analytes are terpenes, at least one secondary clade is obtained based on scoring one or more of the terpenes for one or more therapeutic effects, where the plant strains that are members of the clade share the same average therapeutic profile. In embodiments, the therapeutic effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects. In certain embodiments, at least one therapeutic effect is AChEI and, in embodiments, the terpenes that are scored include one or more terpenes selected from among alpha pinene, eucalyptol, 3 carene, alpha terpinene, gamma terpinene, cis ocimene, trans ocimene and beta caryophyllene oxide. In certain embodiments, at least one therapeutic effect is analgesic and, in embodiments, the terpenes that are scored include one or more terpenes selected from among alpha bisabolol, alpha terpineol, alpha phellandrene and nerolidol.

In certain embodiments, at least one therapeutic effect is on the brain waves and, in embodiments, the therapeutic effect is gender selective. In embodiments, the terpenes that are scored include one or more terpenes selected from terpinolene, (+) limonene, (+) alpha pinene and (+) beta pinene.

In any of the systems provided herein, the number of primary clades can be 3, 4, 5, 6, 7, 8, 9, 10, 11, 12 or higher. In certain embodiments, the number of primary clades is 7.

Also provided herein is method of classifying a plant test sample, based on the classification systems provided herein that are constructed from reference libraries of plant strains, by:
(a) obtaining a measured amount of one or more individual analytes in the test sample;
(b) optionally, (i) comparing the measured amount of at least one individual first analyte to a reference amount of the first analyte, and/or (ii) comparing the ratio of the measured amounts of at least one individual first analyte and at least one individual second analyte to a reference ratio of the amounts of the first analyte and the second analyte, and if the measured amount and/or ratio is different than the reference amount or ratio, identifying the plant sample as an outlier and excluding the plant sample from the classification system;
(c) normalizing the measured amount of each of the one or more individual analytes, thereby providing normalized individual analyte levels;
(d) obtaining an abundance profile of analytes for the test sample, wherein the abundance profile comprises the normalized individual analyte levels;
(e) comparing the abundance profile of analytes of the test sample to the average central value of the abundance profile of analytes of each of the classification systems provided herein, thereby providing a comparison; and
(f) based on the comparison, assigning the test sample to a primary clade selected from among the plurality of primary clades, thereby classifying the test sample

In certain embodiments, the method further includes:
(1) obtaining, for the plant test sample, the identity and/or normalized measured amount of (i) one or more additional analytes, or (ii) a mixture of one or more individual analytes in (a) and one or more additional analytes, where the additional analytes are associated with heredity and/or a known therapeutic effect and wherein the additional analytes are different than the individual analytes in (a);
(2) obtaining one or more profiles selected from among a heredity profile, a therapeutic profile and an abundance profile based on the identity and/or measured amount of (i) or (ii); and
(3) comparing each of the one or more profiles of the test sample from (2) to the average central value of a corresponding profile of each secondary clade of classification systems provided herein, thereby providing a comparison; and
(d) based on the comparison, assigning the test sample to a secondary clade selected from among the plurality of secondary clades, thereby classifying the test sample.

In certain embodiments, the comparison is by Euclidean analysis. In embodiments, the analytes are terpenes, and, in certain embodiments, the test sample is from a Cannabis plant strain.

Also provided herein are methods of breeding one or more plant strains, by:
(i) obtaining a plurality of plant strains or samples therefrom;
(ii) classifying the plurality of plant strains according to the methods of classification of plant strains provided herein;
(iii) based on the classification, identifying one or more plant strains belonging to a primary clade of interest and, optionally, a secondary clade of interest; and
(iv) breeding the one or more plant strains identified according to (iii).

In certain embodiments, the identification in (iii) is of an analyte abundance profile of interest in a primary clade. In embodiments, the analyte abundance profile is one that confers resistance to growth of the one or more plant strains in certain environmental conditions or geographic locations. In embodiments, the analyte abundance profile is one that is favorable for growth of the one or more plant strains in certain environmental conditions or geographic locations.

In certain embodiments of the methods of breeding provided herein, in (iii), one or more plant strains are identified as belonging to a primary clade of interest and further belonging to at least one secondary clade of interest. In embodiments, the identification of the at least one secondary clade of interest in (iii) is of a heredity profile. In certain embodiments, the identification of the at least one secondary clade of interest in (iii) is of a therapeutic profile. In embodiments, the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

In certain embodiments of the methods of breeding provided herein, in (iii), one or more plant strains are identified as belonging to a primary clade of interest and to more than one secondary clade of interest.

Also provided herein are methods of breeding a plant strain that include:
(i) obtaining a plant strain or a sample therefrom;
(ii) classifying the plant strain using any of the classification systems provided herein and/or using any of the classification systems obtained by the methods provided herein;
(iii) based on the classification, identifying the plant strain as belonging to a primary clade of interest and, optionally, a secondary clade of interest; and
(iv) breeding the plant strain identified according to (iii).

In certain embodiments, the identification in (iii) is of an analyte abundance profile of interest in a primary clade. In embodiments, the analyte abundance profile is one that confers resistance to growth of the one or more plant strains in certain environmental conditions or geographic locations. In embodiments, the analyte abundance profile is one that is favorable for growth of the one or more plant strains in certain environmental conditions or geographic locations.

In certain embodiments, in (iii), the plant strain is identified as belonging to a primary clade of interest and at least one secondary clade of interest. In embodiments, the identification of the at least one secondary clade of interest in (iii) is of a heredity profile. In certain embodiments, the identification of the at least one secondary clade of interest in (iii) is of a therapeutic profile. In embodiments, the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity. In certain embodiments, in (iii), the plant strain is identified as belonging to a primary clade of interest and to more than one secondary clade of interest.

In any of the methods of breeding provided herein, in certain embodiments, the analytes are terpenes. In any of the methods of breeding provided herein, in certain embodiments, the plant strain or strains are Cannabis strains.

Also provided herein is a method of cultivating one or more plant strains as a crop, by:
(i) obtaining a plurality of plant strains or samples therefrom;
(ii) classifying the plurality of plant strains according to any of the methods provided herein;
(iii) based on the classification, identifying one or more plant strains belonging to a primary clade of interest and, optionally, a secondary clade of interest; and
(iv) cultivating the one or more plant strains identified according to (iii) as a crop.

In certain embodiments, the identification in (iii) is of an analyte abundance profile of interest in a primary clade. In embodiments, the analyte abundance profile is one that confers resistance to growth of the one or more plant strains in certain environmental conditions or geographic locations. In embodiments, the analyte abundance profile is one that is favorable for growth of the one or more plant strains in certain environmental conditions or geographic locations.

In certain embodiments of the methods of cultivation provided herein, in (iii), one or more plant strains are identified as belonging to a primary clade of interest and at least one secondary clade of interest. In embodiments, the identification of the at least one secondary clade of interest in (iii) is of a heredity profile. In embodiments, the identification of the at least one secondary clade of interest in (iii) is of a therapeutic profile. In certain embodiments, the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity. In certain embodiments, in (iii), one or more plant strains are identified as belonging to a primary clade of interest and more than one secondary clade of interest.

Also provided herein is a method of cultivating a plant strain as a crop, by:
(i) obtaining a plant strain or a sample therefrom;
(ii) classifying the plant strain using the classification systems provided herein or the classification systems obtained by the methods of classification provided herein;
(iii) based on the classification, identifying the plant strain as belonging to a primary clade of interest and, optionally, a secondary clade of interest; and
(iv) cultivating the plant strain identified according to (iii) as a crop.

In embodiments, the identification in (iii) is of an analyte abundance profile of interest in a primary clade. In embodiments, the analyte abundance profile is one that confers resistance to growth of the one or more plant strains in certain environmental conditions or geographic locations. In embodiments, the analyte abundance profile is one that is favorable for growth of the one or more plant strains in certain environmental conditions or geographic locations.

In certain embodiments of the methods of cultivation provided herein, in (iii), one or plant strains are identified as belonging to a primary clade of interest and at least one secondary clade of interest. In embodiments, the identification of the at least one secondary clade of interest in (iii) is of a heredity profile. In certain embodiments, the identification of the at least one secondary clade of interest in (iii) is of a therapeutic profile. In embodiments, the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity. In certain embodiments, in (iii), the plant strain is identified as belonging to a primary clade of interest and to more than one secondary clade of interest.

In any of the methods of cultivation provided herein, the analytes can be terpenes. In any of the methods of cultivation provided herein, the plant strain or strains can be Cannabis strains.

Also provided herein are methods of treatment in which a candidate subject is treated with one or more plant strains or a portion thereof or an extract thereof, by:
(i) obtaining a plurality of plant strains or samples therefrom;
(ii) classifying the plurality of plant strains according to any of the classification methods provided herein;
(iii) based on the classification, identifying one or more plant strains belonging to a primary clade of interest and at least one secondary clade of interest based on a therapeutic profile of the analytes of the plant strains; and
(iv) treating the subject with the one or more plant strains identified according to (iii), or with a portion thereof, or with an extract thereof.

Also provided herein is a method of treating a subject with a plant strain or a portion thereof or an extract thereof, by:
(i) obtaining a plant strain or a sample therefrom;
(ii) classifying the plant strain using any of the classification systems provided herein, or any of the classification systems obtained by the methods of classification provided herein;
(iii) based on the classification, identifying the plant strain as belonging to a primary clade of interest and at least one secondary clade of interest based on a therapeutic profile of the analytes of the plant strain; and
(iv) treating the subject with the plant strain identified according to (iii), or with a portion thereof, or with an extract thereof.

In any of the methods of treatment provided herein, in embodiments, the subject is a human or an animal. In certain embodiments, the portion thereof of the plant is a seed, flower, stem or leaf of the one or more plant strains. In embodiments, the subject is treated with a portion or an extract of the one or more plant strains. In certain embodiments, the treatment is administered orally, topically, or through inhalation. In embodiments, the treatment can be self-administered by the subject and in certain embodiments, the treatment can be administered by an entity other than the subject.

In certain embodiments of the methods of treatment provided herein, the identification in (iii) includes identification of an analyte abundance profile of interest in the primary clade. In embodiments, the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity. In certain embodiments, in (iii), one or more plant strains are identified as belonging to a primary clade of interest and to more than one secondary clade of interest.

In any of the methods of treatment provided herein, in certain embodiments, the analytes are terpenes. In any of the methods of treatment provided herein, in certain embodiments, the plant strain or strains are Cannabis strains.

In any of the classifying methods, methods of assignment of a test sample to a class, methods of breeding, methods of cultivating a plant as a crop, methods of treatment, and other methods provided herein, one or more of the steps of classifying the plant strains can be performed by a machine that includes one or more microprocessors and memory, wherein the memory contains instructions for performing one or more steps of classifying the plant strains one or more microprocessors execute the instructions. In embodiments, the instructions are for classifying one or more plant strains into primary clades and in certain embodiments, the instructions further include instructions for classifying the plant strains of a primary clade into one or secondary clades.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIG. 1 compares the terpene profiles of two strains of Cannabis.

FIG. 2 depicts an example of a terpene profile-based classification obtained by the methods provided herein.

FIG. 3 depicts an example of a flow chart depicting the assignment of a strain sample to a primary clade.

FIG. 4 depicts an example of a flow chart showing the assignment of primary clades into secondary clades based on properties such as heredity (abundances of secondary terpenes) or therapeutic activity (scoring of one or more therapeutic effects).

FIG. 5 depicts the secondary clades (Tier 2).

FIG. 6 depicts an example of 4 different secondary clades within primary Clade 2, based on scoring for different therapeutic effects.

FIG. 7 depicts an example of a weighting factor profile for alpha pinene.

FIG. 8 is a flow chart depicting an example of the overall classification scheme of the methods provided herein.

FIG. 9 is a flow chart depicting an example of how the classification clades are obtained by the methods provided herein.

FIG. 10 depicts a specific example of the flow chart depicted in FIG. 9, where the secondary clades are clustered within the primary clades according to therapeutic activity.

FIG. 11 is a flow chart that depicts an example of how to classify (assign) a test sample based on the clades that have been constructed from a reference library.

FIG. 12 is a flow chart that depicts an example of an overview of how to sub cluster terpenes within the primary clades (i.e., obtain secondary clades).

FIG. 13 is a flow chart that depicts an example of how to assign test samples to secondary clades that are scored for heredity.

FIG. 14 is a flow chart that depicts an example of an overview of how to construct secondary clades based on therapeutic activity.

FIG. 15 is a flow chart that depicts an example of how to assign test samples to secondary clades that are scored for therapeutic activity.

FIG. 16 depicts an example of the dissipation of terpenes in Cannabis samples during storage due to volatility.

FIG. 17 depicts relative terpene abundance based on the analysis of 1683 Cannabis samples.

FIG. 18 shows the maximum concentration of each terpene depicted in FIG. 17.

FIG. 19 depicts the distribution of the most abundant terpenes selected for analysis as primary terpenes in a primary clade classification.

FIG. 20 depicts Kmeans cluster analysis of the primary terpenes selected based on FIGS. 18 and 19.

FIG. 21 depicts the primary clades identified based on the primary terpene profiles clustered as shown in FIG. 20.

FIG. 22 depicts Kmeans cluster analysis, within the limonene dominant primary clade, of secondary terpenes having sedative effects.

FIG. 23 depicts Kmeans cluster analysis, within the alpha pinene dominant primary clade, of secondary terpenes having sedative effects.

### DETAILED DESCRIPTION

Terpenes

Terpenes are aromatic compounds that are a class of unsaturated compounds found in the essential oils of many plants. The molecular structures of terpenes consist of five carbon isoprene units. Mono terpenes contain 2 isoprene units, sesquiterpenes contain 3 isoprene units, and diterpenes contain 4 isoprene units. Terpenes are synthesized in the plant genome by terpene synthase enzymes (TPS). These aromatic compounds create the characteristic scent of many plants, such as cannabis, pine, and lavender, as well as fresh orange peel. The fragrance of most plants is due to a combination of terpenes. Terpenes play central roles in plant communication with the environment, including attracting beneficial organisms, repelling harmful ones, and communication between plants. In nature, these terpenes can protect the plants from animal grazing or infectious germs.

Terpenes also can offer health benefits to animals, including humans. Terpenes and essential oils have been studied over decades as remedies for a variety of medical conditions and have been found to have a wide range of biological and therapeutic properties. For example, terpenes are known to have antioxidant, anti-inflammatory, antibacterial, antiviral, anti-anxiety, antinociceptive, analgesic, antihypertensive, sedative, antidepressant, neuro protective and gastro protective properties. More recently, researchers have looked at the individual terpenes in essential oils, to understand which terpenoids might be contributing to their overall biological and medical properties. Terpenes in essential oils can either exert their individual effects in the oil or they can operate synergistically or agonistically with other oil constituents, giving rise to the term “entourage effects.”

Terpenes in Cannabinoids

In Cannabis plants, such as C. sativa, more than 100 terpenes have been identified. Monoterpenes and sesquiterpenes are responsible for most of the odor and flavor properties of C. sativa, meaning that variation in terpene content is an important differentiator between cultivars. Therefore, there has long been interest from breeders in creating cultivars with particular terpene profiles. Further, there is a growing body of preliminary evidence that terpenes play a role in the various effects of C. sativa on humans, either directly or by modulating the effect of the cannabinoids, implying that medical C. sativa breeding likely will include terpene targets. Therefore, a method of classifying plant strains according to terpene content can facilitate the identification of plants that have the desired phenotypes/characteristics for agricultural, industrial or medical uses.

Terpenes can be analyzed (e.g., identified and/or quantitated) for classification according to the methods provided herein, and for subsequent use of the classification methods/systems in, e.g, methods of breeding, cultivation or therapy, by several techniques. These techniques include, but are not limited to, gas chromatography with a flame ionization detector (GC-FID), gas chromatography-mass spectrometry (GC-MS) and headspace solid-phase microextraction (HS-SPME) in conjunction with GC-MS.

Classification of Plant Strains into Clades Based on the Amount and Type of Terpene Content.

Provided herein is a method of classifying plant strains based on the amount and/or types of terpenes that are present in the strains. Samples (e.g., flower, whole plant, leaf, stem or combination thereof or extract thereof) from a library of plant strains are obtained, processed according to the methods known to those of skill in the art and described herein (e.g., in Example 1) and their terpene chemovars (chemotype or profiles) classified into primary and, optionally, secondary, tertiary or other higher order clades according to the methods provided herein. The word “sample,” as used herein, refers to a plant strain or any portion or extract thereof that contains all or a fraction of the analytes (e.g., terpenes) that are analyzed according to the methods provided herein.

In embodiments, for developing the general cluster model, sample collection for the library can be conducted over all seasons and under a variety of growing conditions to include strains that are grown indoors, in the greenhouse, and outdoors. Terpene profiles of the same cloned genetics can sometimes change based on agricultural and/or geographic conditions, making inclusion of multiple geographic areas and grow culture methods desirable in certain embodiments. In embodiments, for the classification methods provided herein, replicate samples of high similarity within a strain name can be included once to reduce redundancy. In certain embodiments, samples of differing phenotypes that arise from strain chemovar heterozygosity or environmental conditions can be include for analysis according to the methods provided herein. For example, in the library of samples analyzed in Example 1, the data base included an example for each identified strain with up to three chemovar phenotypes that differ in the 5 most abundant terpenes. Once the library of strains is classified according to the methods provided herein, a test sample can be assigned to one or more clades identified by classifying the reference library of strains.

In a first tier of classification (used interchangeably herein with primary classification), the plant strains are grouped into familial clades according to the relative abundances of terpenes that are present in the strains. As used herein, the term “clade” refers to a familial group of plant strains that is constructed based on one or more shared features. For example, in a first tier of classification according to the methods provided herein, the plants are grouped into clades based on shared relative abundances of terpenes. Any number of terpenes can be selected as the primary terpenes used to group the plant strains in the first tier of classification (primary classification), according to their relative abundances. The terpenes analyzed in the first tier are termed the primary terpenes. For example, in Cannabis, there are over 100 terpenes and all their relative abundances could be measured in the plant strains and used to classify them into familial clades in the primary classification (based on relative abundances of all the terpenes). The more the number of terpenes whose abundances are measured for the first tier or primary classification, the more the number of clades that can be present due to the differences in terpene abundance profiles between the strains. If too many clades are present, differences between them can be difficult to distinguish due to overlapping terpene abundance profiles. A smaller number of primary terpenes that generally are present at non trace levels and that generally are present in moderate to high abundance often is needed in order to reliably obtain distinguishable primary clades. The remaining terpenes of interest that are present in smaller amounts (termed “secondary terpenes”) can optionally then be further classified within each of the primary clades in second, third, fourth or higher tier analyses according to their agricultural, industrial or medical properties.

Thus, in certain embodiments, the primary terpenes whose abundances are measured for the first tier of classification (primary classification) are the dominant terpenes in the strains. The term “dominant terpenes,” as used herein refers to terpenes that are present in an amount that is at least or about 3%, 4%, 5%, 6%, 7%, 8%, 9%, 10%, 11%, 12%, 13%, 14%, 15%, 16%, 17%, 18%, 19%, 20% or more by weight of the total amount by weight of all terpenes recovered from the plant sample (e.g., whole plant or a part such as flowers, leaves, stems or a combination thereof). In embodiments, the dominant terpenes are the terpenes that are present in an amount of between 9% to 10%, or at least about 10% by weight of the total amount by weight of all terpenes recovered from the plant sample. In certain embodiments, the dominant terpenes are present as the most abundant terpene in at least one strain of the group of plant strains being classified into primary clades. For example, as shown in Example 1 herein, in measurements on 43 mono and sesquiterpenes of 1683 flower samples from Cannabis representing 375 strain phenotypes, 6 terpenes were identified as dominant: beta myrcene, beta caryophyllene, limonene, alpha pinene, beta farnesene, and terpinolene. At least one strain sample had each of these six terpenes as the most abundant one in the flower.

In embodiments of the methods provided herein, the primary terpenes whose abundances are measured for the first tier of classification (primary classification) include the dominant terpenes in the strain and co-products of the dominant terpenes. The term “co-products,” as used herein, refers to two or more analytes (e.g., terpenes) that are produced simultaneously and/or are present together in the plant at a defined ratio or ranges of ratios. In embodiments, the co-products are present due to genetics, e.g., two or more terpenes that are synthesized by the same terpene synthase enzyme.

For example, as described in Example 1 herein, humulene (alpha caryophyllene), beta pinene, and alpha farnesene are termed “co-products” of beta caryophyllene, alpha pinene, and beta farnesene, respectively, because each set of co-products is produced together, likely due to being catalyzed by the same terpene synthase enzymes in the plant. As shown in Example 1, the 6 dominant terpenes and these 3 co-products (total of 9 terpenes) were used to construct primary clades based on terpene abundance.

In embodiments of the methods provided herein, samples obtained from the plant strains (e.g., whole plant, flower, stem, leaf, etc.) are screened for outliers that are excluded from analysis by the classification methods provided herein. For example, if a plant sample is identified as having lost more than an acceptable threshold of terpene content, e.g., due to volatility (low boiling point and/or high surface area), processing or ageing from storage, such samples can be identified as outliers and excluded from the classification system. Outlier tests can be designed to use ageing and the known co-production of terpenes to exclude the sample profiles that do not conform to the expected genetic co-production of terpenes by TPS (terpene synthase) enzymes. Reasons for failure to conform can include errors in COA (Certificate of Analysis), ageing or sample handling losses of terpenes. For example, some terpenes (e.g., monoterpenes) can be lost during processing due to their low boiling point or high surface area. Criteria for selecting outliers can include one or more of the following:
12) The percentage of decarboxylated tetrahydrocannabinolic acid (THCA) in the sample. Decarboxylated THCA is tetrahydrocannabinol (THC), which is the psychoactive form. The percentage of THC is obtained using the equation: ([THC]/[THCA+THC])×100, where [THC] is the concentration of THC and [THC+THCA] is the total concentration of THC and THCA in the sample. If the THC percentage is greater than 10%, the sample is excluded from the data base due to sample storage, ageing or handling issues which can cause depletion of terpenes.
13) The beta caryophyllene/humulene ratio produced by TPS (terpene synthase) genes has averaged 3.2:1 but a range of 2:1 to 6:1 is acceptable due to analytical error and storage/handling losses and the rest are screened out as outliers.
14) If alpha pinene is greater than 2× (two fold) the limit of quantization, beta pinene must be detected or the sample is declared an outlier as these are co-produced by the TPS genes, with alpha pinene/beta pinene ratios from 0.3:1 to 6:1.
15) If beta pinene is at limit of quantitation (LOQ), alpha pinene must be detected or the sample is identified as an outlier.

Other tests for identifying outliers can include: terpinolene/3-carene ratios at 15:1, with a range from 10:1 to 38:1, terpinolene/alpha phellandrene ratios at 16:1, with a range from 5:1 to 30:1, terpinolene/alpha pinene ratios from 20:1 to 100:1, alpha terpineol/fenchol ratios from 0.3:1 to 2.5:1, terpinolene/gamma terpinene ratios at 50:1, with a range from 20:1 to 120:1 (most of the abundance data is near the limit of detection (LOD), making the range of ratios broader), and terpinolene/sabinene or sabinene hydrate ratio of about 100:1. In embodiments, samples with <0.01, 0.02, 0.03, 0.04, 0.05, 0.06, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1% total terpenes by weight, based on the total dry weight of the sample, can be excluded as outliers prior to the classification.

In embodiments of the methods provided herein, the primary clades obtained by abundance analysis of the primary terpenes, as described above, can further be subjected to classification within each primary clade. Within each primary clade, secondary terpenes can be clustered into secondary clades based on properties other than terpene abundance, such as heredity/ancestry and therapeutic or other biological activity, or combinations thereof. Secondary terpene patterns can also be important ancestry markers, and some are more persistent than most primary terpenes, under variable storage conditions. The first-tier clades assure some similarity within the group of profiles for a more streamlined therapeutic comparison between chemovars. The unknown sensitivities, different therapeutic effects and the tendency of dissipation of the most abundant monoterpenes all support an approach using a simple initial clustering in the first tier into clades, followed by a closer examination of secondary terpenes in the second tier in order to assess the medical effects in absence of large variations in primary terpenes.

The term “secondary terpenes,” as used herein, refers to the terpenes other than the primary terpenes that are classified according to the methods provided herein. Thus, the secondary terpenes are analyzed for clustering within the primary clades. The secondary clades can further be analyzed for clustering in tertiary or higher clades. For example, if the secondary clades are constructed based on heredity, terpenes of the strains within each heredity clade can further be analyzed for medical properties, e.g., sedation, antinociceptive, analgesic and/or antihypertensive properties. In this way, a hierarchical classification system that provides groups of strains that have a set of desired properties can be identified. In certain embodiments, the primary terpenes can be included with the secondary terpenes in the criteria (e.g., therapeutic effects) for secondary analysis. Weighting factors can be used in the secondary or higher clade analyses, e.g., based on potency, to compensate for the greater abundancy of the primary terpenes (often an order of magnitude or higher).

For analyses of the secondary and higher clades, scoring factors can be used, depending on the property (agricultural, industrial, therapeutic effects) being analyzed and depending on the potency of a terpene in relation to that property. For example, for scoring for therapeutic effects, provided below is a Table that summarizes some of the therapeutic activities of several terpenes, and the relative magnitude of the activity (e.g., potent, moderate, mild, no notable effect)

| | | | anti | muscle | anti | antinociceptive |
|---|---|---|---|---|---|---|
| | AChEl | sedative | depressant | relaxant | anxiety | pain blocker |
| Primary | | | | | | |
| Terpenes | | | | | | |
| beta myrcene | no notable | weak | | moderate | | no notable |
| | effect | | | | | effect |
| beta | no notable | no notable | moderate | | moderate | no notable |
| caryophyllene | effect | effect | | | | effect |
| limonene | agonistic | moderate to | moderate | moderate to | moderate to | no notable |
| | | strong | | strong | strong | effect |
| alpha pinene | very strong | agonistic | moderate | | moderate | no notable |
| | (potent) | | | | | effect |
| beta | moderate to | | | | | no notable |
| farnesene | strong | | | | | effect |
| terpinolene | no notable | weak | | moderate | | no notable |
| | effect | | | | | effect |
| humulene | no notable | no notable | | | | no notable |
| | effect | effect | | | | effect |
| beta pinene | no notable | moderate | moderate | | | no notable |
| | effect | | | | | effect |
| alpha | moderate to | | | | | no notable |
| farnesene | strong | | | | | effect |
| secondary | | | | | | |
| terpenes | | | | | | |
| linalool | no notable | very strong | very strong | | very strong | very strong |
| | effect | (potent) | (potent) | | (potent) | (potent) |
| beta ocimene | very strong | | | | | no notable |
| | (potent) | | | | | effect |
| a bisabolol | no notable | no notable | | very strong | | very strong |
| | effect | effect | | (potent) | | (potent) |
| fenchol | no notable | moderate | | moderate to | | no notable |
| | effect | | | strong | | effect |
| alpha | no notable | moderate | | very strong | | very strong |
| terpineol | effect | | | (potent) | | (potent) |
| guiaol | no notable | no notable | | moderate to | | no notable |
| | effect | effect | | strong | | effect |
| camphene | no notable | moderate | | | | no notable |
| | effect | | | | | effect |
| alpha | no notable | no notable | moderate | | | very strong |
| phellandrene | effect | effect | | | | (potent) |
| 3 carene | very strong | | | | | no notable |
| | (potent) | | | | | effect |
| nerolidol | no notable | very strong | | | very strong | very strong |
| | effect | (potent) | | | (potent) | (potent) |
| alpha | very strong | | | | | no notable |
| terpinene | (potent) | | | | | effect |
| eucalyptol | very strong | | | | | no notable |
| | (potent) | | | | | effect |
| eugenol | | | | | | very strong |
| | | | | | | (potent) |

| | | | beta wave | | motor |
|---|---|---|---|---|---|
| | analgesic | alpha wave | boost: | GABA A | stimulation |
| | pain relief | boost: focus | creativity | Modulation | (EPM) |
| Primary | | | | | |
| Terpenes | | | | | |
| beta myrcene | moderate | | | | |
| beta | moderate | | | | |
| caryophyllene | to strong | | | | |
| limonene | moderate | | very strong | | |
| | | | (potent), | | |
| | | | women only | | |
| alpha pinene | weak | moderate | very strong | moderate | moderate |
| | | | (potent), | | |
| | | | women only | | |
| beta | no notable | | | | |
| farnesene | effect | | | | |
| terpinolene | no notable | very strong | | | |
| | effect | (potent), | | | |
| | | women only | | | |
| humulene | no notable | | | | |
| | effect | | | | |
| beta pinene | moderate | | very strong | | |
| | to strong | | (potent), | | |
| | | | women only | | |
| alpha | no notable | | | | |
| farnesene | effect | | | | |
| secondary | | | | | |
| terpenes | | | | | |
| linalool | moderate | | | very strong | |
| | to strong | | | (potent) | |
| beta ocimene | no notable | | | | |
| | effect | | | | |
| a bisabolol | no notable | | | | |
| | effect | | | | |
| fenchol | no notable | | | | |
| | effect | | | | |
| alpha | very strong | | | | |
| terpineol | (potent) | | | | |
| guiaol | | | | | |
| camphene | no notable | | | | |
| | effect | | | | |
| alpha | moderate | | | | |
| phellandrene | to strong | | | | |
| 3 carene | no notable | | | | |
| | effect | | | | |
| nerolidol | moderate | | | moderate to | |
| | to strong | | | strong | |
| alpha | | | | | |
| terpinene | | | | | |
| eucalyptol | | | | | |
| eugenol | | | | | |

In embodiments, the secondary classification can be based on an overall scoring of the therapeutic effects of the secondary terpenes (or the secondary terpenes and weighted primary terpenes). In certain embodiments, the secondary classification can be based on a scoring and/or filtering of a subset of the secondary terpenes (or the secondary terpenes and weighted primary terpenes). For example, the secondary clade construction can be based on scoring and/or filtering of terpenes that effect Acetylcholinesterase inhibition (ACHEI), which enhances cognitive function. The group of active ACHEI terpenes can include one or more of caryophyllene oxide, 3 carene, gamma and alpha terpinenes, eucalyptol, camphor thymol thujone and alpha pinene. In embodiments, limonene and camphor can be included in the scoring and/or filtering, as agonists that negatively impact the ACHEI (acetylcholinesterase inhibition) activity of terpenes such as alpha pinene and eucalyptol. In certain embodiments, alpha pinene can be included in the scoring and/or filtering, as an agonist that interferes with (reduces) sedation by limonene. As another example, secondary clade constructions can be based on scoring and/or filtering of terpenes that have antinociceptive activity, such as one or more of alpha bisabolol, alpha terpineol, alpha phellandrene and nerolidol. The therapeutic scoring can include all terpenes with known therapeutic effects, such as antioxidant, anti-inflammatory, antibacterial, antiviral, anti-anxiety, antinociceptive, analgesic, antihypertensive, sedative, antidepressant, ACHEI, neuro protective and gastro protective properties, or only one therapeutic effect, or a subset of two or more therapeutic effects.

In embodiments, the therapeutic secondary clade classification is scored and/or filtered for effects on brain wave (EEG) activity and in certain embodiments, the effects can further be scored based on gender specific effects on brain wave activity. For example, in one study, inhalation of terpinolene was found to increase relative fast alpha wave activity and decrease mid beta wave activity, generating a relaxed, focused state. Inhalation of (+) limonene, on the other hand, was found to increase relative high beta wave activity which, when subjected to complex tasks, can cause stress, tension and anxiety. Thus, in general, terpinolene can be considered more beneficial as an inhalant when undertaking complex tasks. These effects, however, were found to be gender specific. In women, both terpinolene and (+) limonene increased absolute fast alpha wave activity, generating a relaxed, focused state and (+) limonene additionally decreased relative mid beta wave activity. Thus, women responded favorably to both (+) limonene and terpinolene. Men, on the other hand, showed no increase in alpha wave activity in response to either of the terpenes. With terpinolene, a decrease in relative mid beta wave activity was observed and with (+) limonene, a relative high beta activity increase was observed. Thus, men showed no significant favorable response (no alpha wave activity increase) to either of these terpenes and in fact could experience undesirable effects (stress, tension, anxiety) by inhalation of limonene, which led to an increase in relative high beta wave activity. In another study using (+) alpha pinene and (+) beta pinene, it was found that women highly responded to both the compounds compared to men. In women, absolute alpha wave, absolute beta wave and absolute high beta wave activity significantly (P<0.05) increased during the inhalation of (+) alpha pinene and, in the case of (+) beta pinene, absolute fast alpha wave and absolute high beta wave activities also significantly increased. In men, on the other hand, there was no impact on alpha waves; significant decreases in absolute waves such as theta, beta, low beta and high beta were observed during the inhalation of (+) alpha pinene but there were no significant changes in the absolute waves by inhalation of (+) beta pinene.

In certain embodiments of the methods provided herein, the secondary classification within the primary clades can be based on a heredity scoring. In general, plant strains within each primary clade are expected to contain the most similar genetics in terpene synthases, TPS, due to their similar bulk production of the most dominant terpenes. Differential effects of the less abundant secondary terpenes can then be examined more efficiently and with greater sensitivity within each clade, to obtain more information about the differences or similarities in the genetics. In embodiments, a weighting factor can be used to correct for the effects of processing, ageing, and the like, such as dissipation. In certain embodiments, a reduced set that includes high boiling terpenes present in the strains and is not overwhelmed by the abundant primary terpenes can be used as a final fingerprint for heredity analysis. These terpenes will be very persistent under ageing due to chemical stability under oxidation and high boiling points. Examples of persistent (high boiling) secondary terpenes include, but are not limited to, alpha bisabolol, alpha terpineol, Guiaol, nerolidol, fenchol and linalool. This reduced set vector should be consistent over time and provide reliable additional information for assigning heredity/genetically related strains as well as correlating the genetics with a therapeutic effect.

The number of terpenes of the plant strain samples that can be analyzed according to the methods provided herein, either in a single tier (primary clades, based on primary terpenes) or multi-tier (primary clade and one or more secondary clades, based on secondary terpenes and/or weighted primary terpenes) can be all of the terpenes that are detected in the sample or a fraction of the terpenes that are detected in the sample, e.g., terpenes that are present in more than trace amounts, or any other fraction of terpenes based on abundance (e.g., most abundant terpenes) or other characteristics, such as high boiling points, biological/therapeutic activity, for breeding, for resistance or for favoring growth in an environmental condition or a geographic location, or for therapeutic use and the like.

For example, between 5 to 100 or more terpenes can be classified according to the methods provided herein. The number of terpenes in the library of plant samples used to construct the primary and secondary (or higher order) clades, or in a test sample analyzed for assignment to primary and/or additional clades can be at least or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more terpenes. In certain embodiments, the number of terpenes analyzed according to the methods provided herein are between 10 to 25 terpenes. In embodiments, 20 terpenes are analyzed and in certain embodiments, 17 terpenes are analyzed. In general, the analysis of fewer terpenes according to the methods provided herein can be faster and cheaper and make it easier to view distinct clades; however, a smaller amount of information is obtained about the strains because a smaller fraction of the terpenes in the strains are analyzed. It was found herein that the analysis of between 15-25 terpenes of a library of plant strains, e.g., between 17-20 terpenes, balanced the ease of constructing clades using a smaller number of terpenes with obtaining sufficient information to classify the strains according to desired characteristics including heredity and therapeutic activity.

In embodiments of the methods provided herein, the terpenes that are classified include one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol. Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

Thus, provided herein is a one or, optionally, multi-tier classifier method that can be used efficiently to separate the relative abundances and other properties of terpenes, first naturally by their dominance and/or co-production with dominant terpenes (according to abundance) in the first tier (primary clades using primary terpenes) to construct familial clades or groups of primary terpenes and then in the second and subsequent tiers, further assessed within the primary clades according to ancestry, therapeutic activity and other agricultural, biological or medical uses. This approach represents a method of assessing the terpenoid profiles in a manner that includes the most abundant terpenes yet preserves more subtle information in the less abundant terpenoids. The clade groups are also efficient way to study the “entourage effects” of less abundant terpenes in efficient test designs and group them according to therapeutic activity or other characteristics, such as ancestry or desirable phenotypes/chemotypes for breeding.

In the methods provided herein, the terpene profiles are first assigned to cluster groups or clades. Clades are expected to contain the most similar genetics in terpene synthases, TPS, due to their similar bulk production of the most dominant terpenes. Differential effects of less abundant terpenes can then be examined more efficiently within each clade with the appropriate clinical testing. The information in this smaller within clade profile data (secondary, tertiary and greater clusters) could be important due to differing therapeutic effects and potencies of different terpenes. Some enzyme inhibitor and receptor channel modulation effects will not be linear with concentration, adding to the complexity of therapeutic assessment. The approach described here simplifies the interpretation of terpene entourage effects in clinical studies by permitting the observation of a few changes in terpenes of lower abundance while the most abundant terpenes are consistent within each primary clade. Provided herein is a single or multi-tiered clade or system for evaluation of plant strains and strain phenotypes based on plant terpene profile content and the effects of the terpenes. The method uses a separation where first tier clade groups are defined by their most abundant “dominant” primary terpenes (and additionally including terpenes co-produced with one or more of the dominant terpenes) and the second-tier separation excludes or de-emphasizes the primary terpenes inside each clade in favor of secondary terpene profile information. In the absence of individual scaling, the most abundant terpenes are most influential in clustering by their greater variation in abundance. In the second tier, sub-clustering of the less abundant secondary terpenes can independently be conducted within each clade, to identify terpene based genetic markers and secondary terpene therapeutic, agricultural and industrial or other effects.

If terpenoid activities were only a simple function of concentration and all terpenes had the same activities, an unweighted clustering analysis of a single tier or another non-tiered clustering approach might gather all the information necessary. But since terpenoids can have more than an order of magnitude variation in quantified bio activity of terpenes, the most abundant terpenes can be expected to dominate the initial unweighted clustering regardless of their therapeutic activity.

Less abundant, but potentially more therapeutically active, secondary terpenoids do not have much impact on distances in the initial top tier clustering. But in the sub clustering in subsequent tiers, lower abundance terpenes (secondary terpenes) can be more influential by exclusion or down weighting of the most abundant ones (primary terpenes classified into primary clades). This allows the relative abundances of the less abundant terpenes to be examined without quantifying weights for different secondary terpenes. It is expected that the expression of Terpene synthase enzyme activity in the plant gives rise to the plant terpene abundance profile at harvest, though curing and storage effects can alter profiles, particularly in the volatile monoterpenes. Excluding or down weighting (e.g., for dissipation effects or for reduced potency, e.g., in a sub-clustering for therapeutic activity) these volatile monoterpenes from the clade sub-clustering into secondary clades or beyond can allow for removal of the highest impact storage and handling contributions in the heredity groupings.

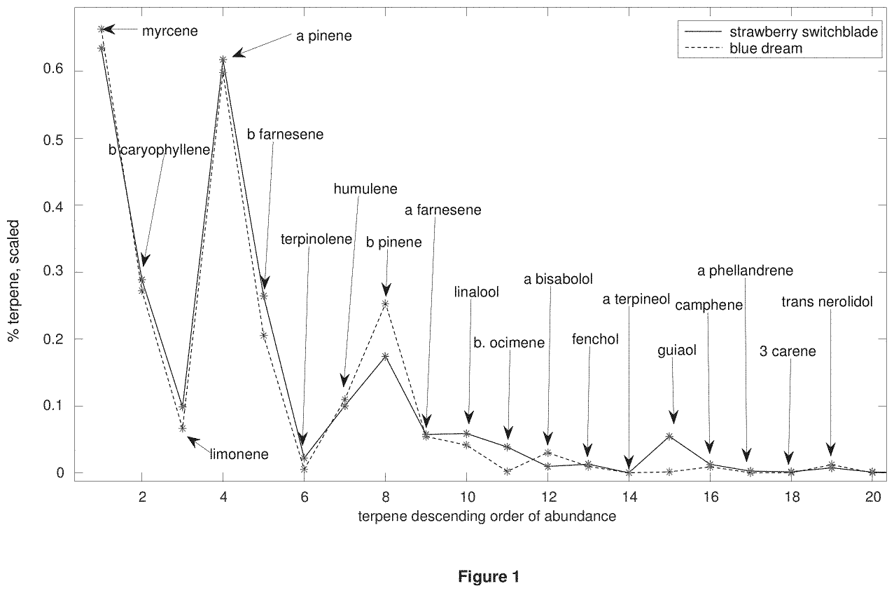

Clade representations obtained according to the methods provided herein can permit the investigation of secondary terpene effects among samples with a similar distribution of primary terpenes, and to define systematically differing groups of primary terpenes that can be compared and contrasted for their effects. For example, in FIG. 1, the terpene profiles of the Cannabis strains “Blue Dream” and “Strawberry Switchblade” are plotted.

As FIG. 1 shows, the two strains are highly similar in the profiles of the dominant terpenes (more abundant terpenes), which could indicate the potential for common effects, such as therapeutic effects, of these two strains. The two strains however differ in their beta pinene, beta ocimene, alpha bisabolol and guaiol content, as seen in FIG. 1. Therefore, if therapeutic differences are present between the two strains, they could be attributed to the variation of these less abundant terpenes, particularly if they have high potency. Thus, examining the secondary terpenes in the absence of or in the weighted presence of the primary terpenes can provide useful information about the different applications of even seemingly very similar strains.

While the methods provided herein are exemplified using terpenes, those of skill in the art will understand that the principles of the invention can be applied to one or more of any of the compounds that are components of the chemical profiles of plant strains, including, but not limited to, monoterpenes, sesquiterpenes, diterpenes, sesquiterpene lactones, flavonoids, carotenoids, cannabinoids, or any combination thereof. In embodiments, the compounds provide information about lineage or heredity. In certain embodiments, the compounds render the plant strain resistant to or conducive for growing under certain environmental conditions, or in certain geographic locations. In certain embodiments, the compounds have biological or therapeutic activity. In embodiments, the plant strains that are analyzed and classified according to the methods provided herein are Cannabis strains.

Statistical Methods

Overview

Certain statistical terms used in the analyses described below are as follows:
Centroid: a 1×n vector containing the average analyte value for all samples within a clade or cluster.
Vectors are in boldface lower case e.g., a, scalars are in lowercase, e.g., a, and matrices are in bold uppercase, e.g., X.
There are n analytes (e.g., terpenes) indexed by the subscript i, measured for each sample
There are j clades with j centroid vectors, each centroid is a 1×n analyte vector of mean values
Scores, s is a score vector (1× number of pc's kept) from PCA decomposition of a, the sample analyte vector
a=sp^{t} where s is the score projection onto p the PCA coordinate axes, t is the transpose of the vector

As discussed above, a set of primary terpenes, which represent the most abundant terpenes and, optionally, terpenes that are present as co-products of one or more of the most abundant terpenes, define initial clustering of the terpenes from samples of a library of plant strains into the first tier of cluster groups or “clades” (primary clades). Outlier samples due to the effects of dissipation, ageing, processing and the like can be identified as described herein and set forth in the examples, can be excluded or weighted prior to the primary classification.

The secondary terpene set, whose abundances relative to the primary terpenes can be less by one or more orders of magnitude, can have several terpenes that exhibit therapeutic activity in areas that may either support or are not exhibited by many of the primary terpenes. In addition, while they generally are present in much smaller amounts than the primary terpenes, their potency could be high, as therapeutic dosages often can differ by as much as two orders of magnitude. Secondary terpene patterns also can be important ancestry markers, with some being more persistent (e.g., less volatile) than many primary terpenes under sample storage conditions. This supports embodiments of the method in which the more abundant primary terpenes are separated out in a primary classification before fine tuning the classification based on the effects of the secondary terpenes (sub-clustering into secondary or other higher order clades).

The primary clades can provide a broad classification into a few clusters or groups, based on the most abundant terpenes of the plant strains. The terpene profile of a test plant strain sample readily can be screened against the primary clades, which provide an initial simple classification, and the test sample can be assigned to a primary clade based on a vector distance to the clade centroid. If a test sample cannot be assigned to a primary clade based on distance, additional strains can be added to the library of plant strains and classified to obtain additional clades that are a closer match to the test sample.

The less abundant secondary terpenes can then be sub-clustered into secondary, tertiary or other higher order clades, based on the information desired (e.g., ancestry/heredity, therapeutic activity, resistance to or favoring an environmental condition or a geographic location). Weighting schemes can be used to limit the impact of storage and handling on terpene chemovar (chemotype, based on terpene profile) or ancestry identification and to predict sample storage and ageing impact on therapeutic effects. Alternately, the less abundant terpenes can be examined separately from the more abundant volatile primary terpenes. If terpene “A” dissipates rapidly but the therapeutic effects do not change appreciably, the therapeutic classification should reflect this consistency with dissipation of terpenes. If, for example, the therapeutic activity to be examined in the secondary classification is antinociceptive pain relief, the powerful antinociceptive pain relievers of trans nerolidol, alpha phellandrene, alpha terpineol, and alpha bisabolol will likely have more impact than the primary terpenes like myrcene and limonene in storage, which can undergo dissipation. The known individual therapeutic effects can be used to weight/score expected therapeutic utility by weighted (0,1) and scored effects both with primary terpene therapeutic scoring in the first tier, and with secondary terpene scoring in the second tier. The scores of both tiers can then be combined to form an array of medical effects for the terpene profile of a particular strain. Interactive effects of terpenes, synergy or agonistic effects can be analyzed using mixture models or factor analysis of therapeutics outcomes in clinic. Within each clade, a response surface modeling, RSM, can be used to estimate the nature of these non-additive effects. The clade separation obtained by the methods provided herein allows for more simplicity in the study of synergistic and agonistic effects of terpenes in plants, by providing a broad primary clade classification based on the more abundant terpenes; within the broad primary clades, properties such as heredity and therapeutic contributions of the less abundant but often just as or more informative (about heredity or therapeutic properties of a plant strain, e.g.) secondary terpenes can be analyzed by sub-clustering (into one or more secondary clades or other higher order clades).

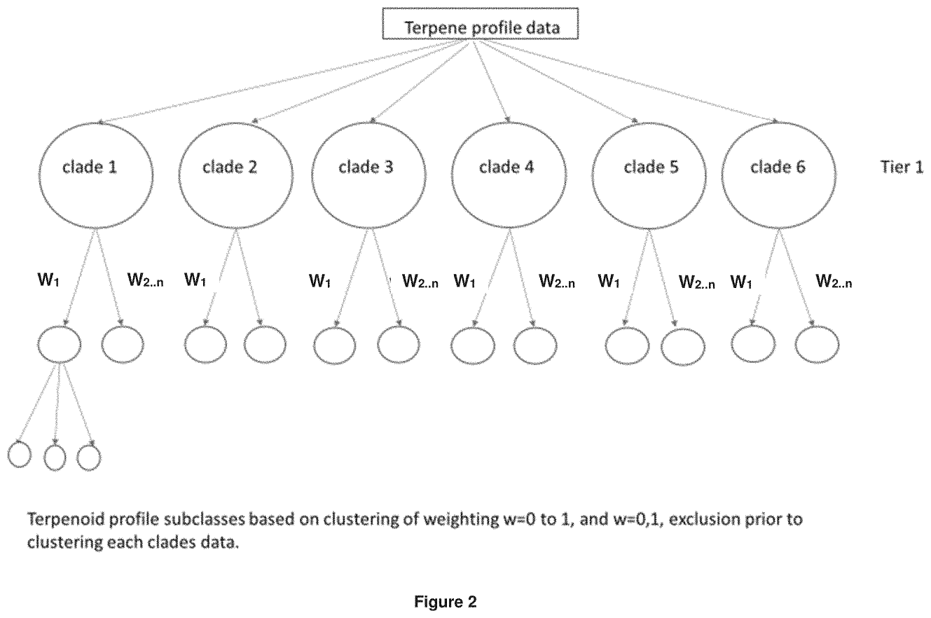

An example of the basic classification structure is depicted in FIG. 2. FIG. 2 depicts 6 primary clades obtained by classifying the primary terpenes of strain samples of a library. As further shown, each of the primary clades can then be sub-clustered into secondary clades based on factors such as heredity/ancestry, agricultural use (for breeding, cultivating a crop, etc.) or therapeutic use. As FIG. 2 also depicts, the secondary clades can further be sub-clustered into tertiary or other higher order clades according to additional desired factors.

Thus, the tiered system provides a simple yet comprehensive way to classify strains according to their terpene profiles. Kmeans clustering can be used to divide the first tier of clades, and in the second tier it is used to cluster within clades. Clustering within clades can use the whole set of terpenes, the secondary terpenes and/or Sativa/Indica terpenes for heredity interpretation or a defined set of terpenes that are expected to produce the desired medical effects. For example, in evaluating sedation, neutral terpenes that are non-sedative can be excluded or de-weighted giving rise to emphasis of terpenes with known sedative action in computing the therapeutic scoring. In embodiments, terpenes that have no known AChE inhibition activity can be excluded from the analysis on memory/cognition therapeutics in scoring chemovars. Weighting or exclusion templates can be used to examine groupings of individual medical effects between strains or expressed genetics of the TPS genes. Distances from the class centroid in the clade groupings can be computed by Euclidean distance (dist) in Equation 1, or by a weighted distance (Wdist) given in Equation 2, with abundances aᵢ and the cluster abundance centroid a_{c}.

dist=[(a₁−a_{c})²+(a₂−a_{c}²+(a₃−a_{c})²+ . . . (aₙ−a_{c})²]^{1/2} (1)

Wdist=[w₁(a₁−a_{c})²+w₂(a₂−a_{c})²+w₃(a₃−a_{c})²+ . . . wₙ(aₙ−a_{c})₂]^{1/2} (2)

There is a potential for defining a weighting set, wᵢ>0, for therapeutic comparison between chemovars.

For an abundant mono terpene with a lower boiling point, the sample concentration variation due to storage and handling could be large in computed distances with Equations 1 and 2, when compared to the small concentration variations of the more persistent terpenes arising from TPS genes. With a bottom up agglomerative clustering method, the closest distance or terpene ratios can be significantly impacted by storage and handling, which leads to tree agglomerations that can be masked by storage and handling effects of volatile or reactive primary terpenes rather than reflecting therapeutic effects or ancestry. In certain embodiments, provided herein are methods in which reduced weighting or exclusion of the most abundant volatile terpenes that would be impacted the most by handling and storage conditions is employed. In embodiments, running parallel assessments of weighed and unweighted terpenes can also have value in interpreting clinically tested therapeutic groupings. This approach can allow for more relevant groupings within each clade that are related to medical effects and heredity. The weight groupings can emphasize specific effects such as anti-anxiety, energizing effects, pain relief, sedative effects, cognitive effects, EEG activity, gender-specific effects and anti-depressant effects. As is known to those of skill in the art, the independent effects of plant terpenes common to Cannabis reveal a wide range of reported medical effects, from pain relief and antimicrobial activity to memory and cognitive stimulation. In a first approximation, some of these individual effects could be used to weight or include (w=0 or w=1) terpenes and group them according to the targeted therapeutics. Weights also can be adapted to reflect the entourage (cumulative or synergistic) therapeutic effects.

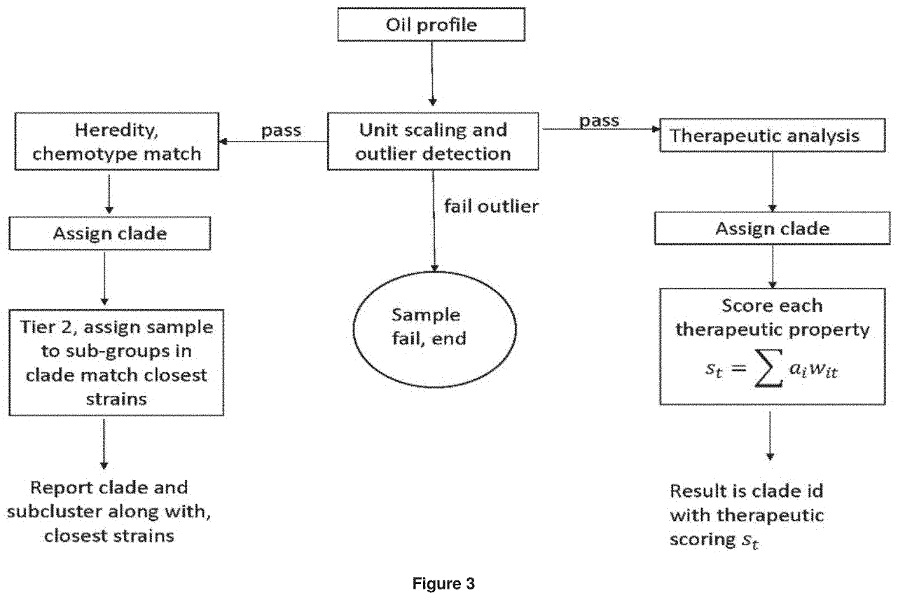

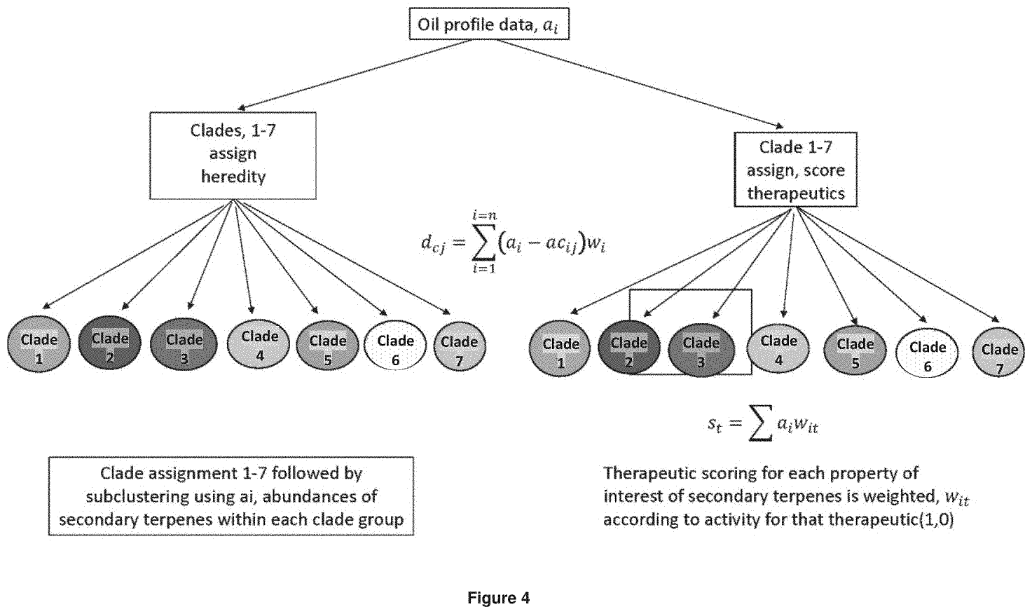

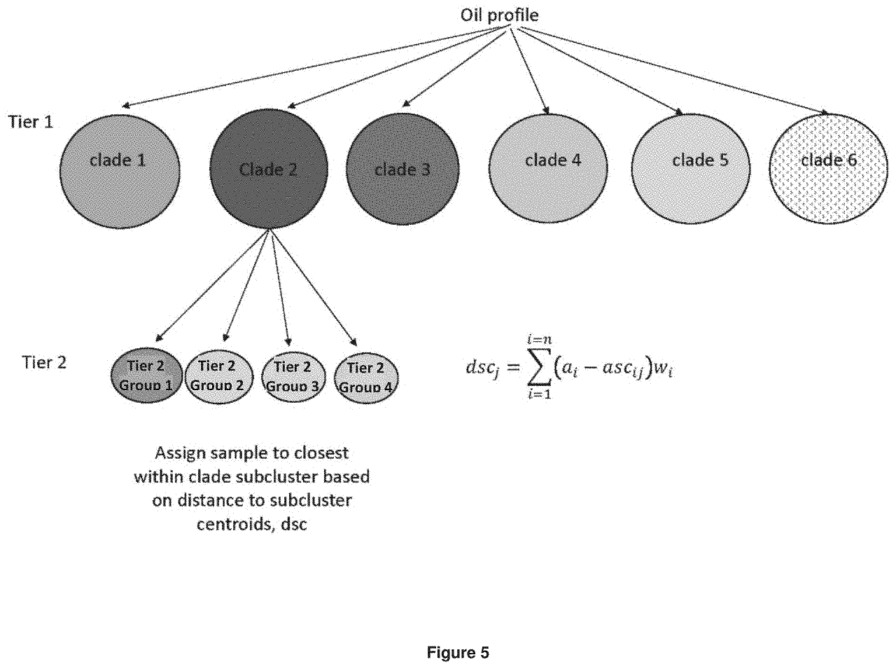

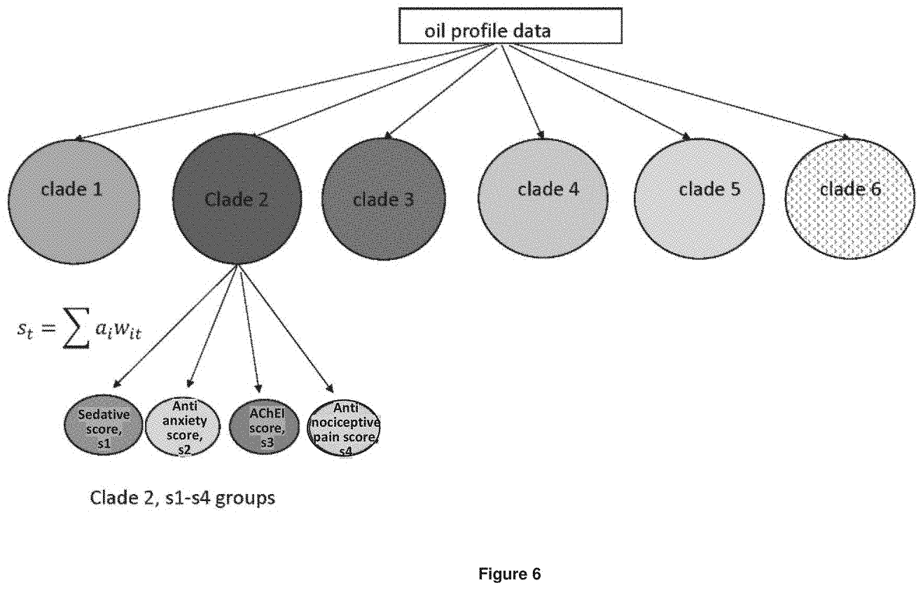

FIG. 3 is an example of a flow chart depicting the assignment of a strain sample to a primary clade. As the flow chart depicts, outliers can be removed prior to the analysis. FIG. 4 is an example of a flow chart depicting the assignment of primary clades into secondary clades based on properties such as heredity (e.g., abundances of secondary terpenes) or therapeutic activity (e.g., scoring of one or more therapeutic effects). FIG. 5 depicts the secondary clades (Tier 2). Test samples can first be assigned to a primary clade based on the closest distance measured to a primary clade centroid and then to a secondary clade within the primary clade based on the closest distance measured to a secondary clade centroid. FIG. 6 depicts an example in which 4 different secondary clades are assigned within primary Clade 2, based on scoring for different therapeutic effects.

Methods

Known terpene concentration profiles of the library of plant strain samples can be used for the analysis. Alternately, stock calibration solutions can be prepared for the number of terpenes desired to be included in the analysis, a calibration developed and applied to all sample data to generate each sample terpene concentration profile. For example, if the terpene profiles of the strain samples contain concentration data for n terpenes, 1×n vector a defines the Y terpene concentrations of each sample, aᵢ. This vector of n terpene concentrations is defined as the strain “terpene profile” or strain chemovar profile.

Preprocessing

Preprocessing includes normalization of the terpene vector profile to unit length.

Normalization of Sample Terpene Profiles (e.g., Library Used to Build Clades)

Each sample vector, a (vectors in bold), is normalized to unit length

Each sample is represented by a terpene vector a of n terpene concentrations, aᵢ, as in Equation (A). Two methods of scaling that have been tested include fractional terpene composition as in Equation (C), using the terpene vector a in Equation (A) and the sum of its vector elements in Equation (B) to obtain the terpene fraction.

a=[a₁a₂a₃ . . . aₙ] (A)

sum(a)=[a₁+a₂+a₃+ . . . aₙ] (B)

a_{pct}=100*(a/sum(a)) (C)

The second scaling method that can be used is scaling by division with the Euclidean norm as in Equation (D)

Norm(a)=[(a₁)²+(a₂)²+(a₃)²+ . . . (aₙ)²]^{1/2} (D)

a_{pct}=a/Norm(a) (E)

As an example, if the sample vector a=[1, 1, 3, 5, 2, 1, 5], then the % norm1(a)=(a/sum(a))*100 (note the times 100 is for % and % is used for clarity as fractions are small decimals)

Calculation of % Norm1(a):

% norm1 (a)=(a/(1+1+3+5+2+1+5))*100=(a/18)*100=[5.56 5.56 16.67 27.78 11.1 5.56 27.78] represents a vector whose elements are the percentages of each terpene with respect to the total sum of terpenes. These percentages are used in therapeutics to look at the % of the total terpenes with a special property, e.g., sedation.

The alternate normalization is the norm2(a)=a/[(a₁)²+(a₂)²+(a₃)²+ . . . (aₙ)²]^{1/2}

Calculation of norm2(a), the Euclidean or second normalization of the vector a.

For the above terpene profile sample vector a, that would be

[1, 1, 3, 5, 2, 1, 5]/((1)²+(1)²+(3)²+(5)²+(2)²+(1)²+(5)²]^{1/2}

Thus norm2(a)=[1 1 3 5 2 1 5]/(1+1+9+25+4+1+25)^{1/2}=[1 1 3 5 2 1 5]/8.12 norm2(a)=[0.1232 0.1232 0.3695 0.6158 0.2463 0.1232 0.6158]

After normalization of terpene profiles, a principal component analysis (PCA) can be used for dimensional reduction of library data before input to the clustering algorithm for clade development. The PCA of A, the original normalized library data matrix of scaled abundances with m sample rows and n terpene columns is decomposed into an m by n scores matrix T and by an m by n loading matrix P.

A=TP^{t} (F)

Where P^{t} denotes the transpose of the loading matrix P. The PCA yields a matrix of m samples by n, the maximum number of terpene scores, as the number of columns in the matrix T. A notably smaller number of scores columns, v, is selected from the first v component scores. This number v replaces the n terpenes in the a vector with a t vector of v score columns. For example, in the analysis described in Example 1, for 43 terpenes analyzed (n=43), the library data did not appear to need more than 11 scores (v=11) as 99% of the library matrix variance was captured in those 11 scores. This should represent an advantage, reducing 43 to 11 variables but false hits or misses on low level terpenes can create a mixing of non-normal “noise” in the PCA that could be a disadvantage. As laboratory errors are reduced and the noise model tends towards multinormal in measurement error, a PCA will have advantages in dimension reduction.

For this illustration, we report on the use of scaled inputs a, as opposed to scores t. The first “v” scores are included in the analysis and the later scores associated with small variance and noise are excluded from the terpene score library and sample matrix. Selection of the number of scores can be performed by methods known to those of skill in the art. The loading matrices, P, are used to convert all new samples into the score space by T=AP. When data complexity rises in the future due to extension of detection limits and addition of new species to the terpene profile, PCA can provide greater clarity in the clustering structure. For this illustration, we use the normalized concentrations directly for input into the clustering algorithm. Normalized chemical concentrations are easier to interpret in terms of the analysis of clustering group terpene profile contents.

Data that have been analyzed to date with and without PCA provide similar results, but differences could occur as detection limits increase. The analysis provided below is with the use of a, the vector of normalized abundances, but it could be substituted by t, the scores vector in each expression.

Clade Assignment Calculations

Distances are used as a similarity measure to assign samples to clades which are each represented by a class mean (centroid) vector. The distances of the sample profile to each of the clade centroids is computed and then the minimum value determines the clade membership.

Clustering with Kmeans

The full library data can next be subjected to a Kmeans cluster analysis for a desired number of clusters, e.g., between 1, 2 or 3 clusters to 4, 5, 4, 5, 6, 7, 8, 9, 10, 11, 12, 13, 14, 15, 16, 17, 18, 19, 20 or more clusters. In embodiments, between 3 to 10, 11, 12, 13, 14 or 15 clusters are analyzed. In certain embodiments, between 3 to 12 clusters are analyzed. An elbow method can be used to examine the optimal number of clusters. Because Kmeans is an optimization, trapping in local minima is possible. To get at the global minima, the algorithm can be run multiple times from different initializations. For example, for the Kmeans analysis described in Example 1, the algorithm was run 100× from different initializations for each cluster number from 3 to 12. The boxplot results are analyzed for the elbow point. For example, in Example 1, the boxplot shows that an elbow point occurs at k=7. The Kmeans solution with the lowest sum of within cluster distances can be saved as the cluster center solution, the clade centroid, aₘ for m=1:Z defining the Z centroid vectors (Z=7 in Example 1) aₘ that define the mth clade.

aₘ=[a₁ₘ+a₂ₘ+a₃ₘ+ . . . aₙₘ] (G)

Distance Calculations

Kmeans classes are defined during the optimization by assigning each library sample to the closest centroid. Thus, the clades are exclusive and non-overlapping. The solution of class membership then determines the centroid as the average of all class members. After the centroid vectors of the clades are established, distances from each of the centroids (7 in Example 1) to the test sample of interest are calculated and the minimum distance is selected as the class membership of the test sample. The centroid vectors a_{d} to a_{cZ} (Z=7 in Example 1) define the centers of the clades from which all distances to new and existing samples can be calculated. The distance dc from normalized sample terpene profile a=[a₁ a₂ a₃ . . . aₙ] to clade 1 centroid

a_{c1}=[a_{c11}a_{c12}a_{c13} . . . a_{c1n}] (H)

is obtained by an unweighted(wi=1) or weighted sum of the squared differences of each element in the vector.

dc₁=[w₁(a₁−a_{c11})²+w₂(a₂−a_{c12})²+w₃(a₃−a_{c13})²+ . . . wₙ(aₙ−a_{c1n})²]^{1/2} (I)

for no weighting of analytes, wi are all equal to 1. Otherwise weights can be established using a priori knowledge, including physical and biological properties, about terpenes of interest.

Secondary Clade Clustering and Test Sample Assignment

After clade membership is determined, the second-tier (secondary clade) clustering within each primary clade can be used to further describe properties such as heredity, agricultural and therapeutic properties. In the second tier, a subset of less abundant terpenes can be used to cluster, since the principal terpenes are common to the primary clade. In embodiments, some of the principal terpenes that are less similar in the primary clade can be used in the secondary clustering. Any primary terpenes that are used are weighted down so as not to disrupt information due to the less abundant secondary terpenes. The same clustering and distance assignments are used in the second tier, except that they do not include the dominant terpenes common to the clade, or they use some of the primary terpenes with smaller weights.

Distance to Clade Centroid Determination for Clustering in Secondary Clades:

As an Example, for the sample vector a=[1, 1, 3, 5, 2, 1, 5] above:

a=[1, 1, 3, 5, 2, 1, 5]=[a₁, a₂, a₃, a₄, a₅, a₆] the (normalized) comma separated sample terpene profile vector;

c=[1, 1, 3, 5, 2, 1, 5]=[c₁ c₂ c₃ c₄ c₅ c₆ c₇] The clade centroid vector (there are 7 clades each with a centroid vector);

distW=[w₁(a₁−c₁)²+w₂(a₂−c₂)²+w₃(a₃−c₃)²+ . . . wₙ(aₙ−cₙ)²]^{1/2} (I)

dist is distance from sample to the clade centroid vector and distW is the weighted distance, where:

wi are weights from 0 to 1 that can be used to compensate for storage, volatility, potency/effects

if all weights, wi=1, the equation reduces to Euclidean or the 2^{nd} norm distance.

dist=[(a₁−c₁)²+(a₂−c₂)²+(a₃−c₃)²+ . . . (aₙ−cₙ)²]^{1/2}

dist=[(1−0)²+(1−0)²+(3−1)²+(5−3)²+(2−3)²+(1−2)²+(5−3)²]^{1/2}

dist=[(1)²+(1)²+(2)²+(2)²+(−1)²+(−1)²+(2)²]^{1/2}=(1+2+4+4+1+1+4)^{1/2}=(16)^{1/2}=4

distance calculations are the same whether it is from one sample to another or to the terpene profile of the sample to any centroid. Secondary clade distance evaluations can include all terpenes (e.g., primary terpenes or weighted primary terpenes and secondary terpenes or any fraction thereof) or only secondary terpenes in the a.

Scoring for Therapeutics in the Secondary Terpenes

Therapeutic scoring can be used to assess the expected therapeutic effects of the terpene set. In certain embodiments, only the secondary terpenes or a subset thereof are used to evaluate the therapeutic activity, depending on the therapeutic indication of interest. In certain embodiments, the secondary terpenes or a subset thereof can be scored for their effects on brain wave (EEG) activity. In embodiments, the secondary terpenes or a subset thereof can be scored for gender specific therapeutic effects. In embodiments, primary terpenes or weighted primary terpenes can be included in the scoring.

In one example, the secondary terpenes can be scored for therapeutic activity such as AChEI cognitive support, sedation, muscle relaxation, anti-anxiety, analgesic pain relief, antinociceptive pain blocking, anti-inflammatory activity, expectorant activity and bronchodilation activity. Similar scoring methods can be used to analyze other properties, such as heredity/ancestry and agricultural use (e.g., screening profiles for in breeding or outcrossing, resistance to or favoring an environmental condition or a geographic location, and the like). Initial scoring without dosing could be 1,0, i.e., present or not present. The sum of all like properties could also be represented. For example, % sedative content in secondary terpene set would just involve summing the percentage of all known sedatives in the sample. For example, if a strain has notable levels of linalool, fenchol, alpha terpineol, nerolidol and camphene, there are 5 sedatives in the secondary terpene set so the score could be ‘5’ or the sum of all sedative terpene percentage abundances divided by all terpenes in the second set. A percentage is attained by multiplication by 100.

Example of sedative scoring: sedative template, yes=1 and no=0, not sedative.

Template t=[1, 0, 0, 1, 0, 1] which indicates secondary terpenes 1, 4 6 are sedative while 2, 3 and 5 are not sedative

terpene abundances(normalized) a=[0.2, 0.4, 0, 0.3, 0.1, 0];

The sedative score is defined by a vector inner product by ai to get therapeutic score and summed up.

Score=t×a (inner product)=(t₁×a)+(t₂×a₂)+(t₃×a₃)+ . . . (tₙ×aₙ) for both t and a as 1 by n vectors

Score=(1×0.2)+(0×0.4)+(0×0)+(1×0.3)+(0×0.1)+(1×0)

Score=0.2+0+0+0.3+0=0.5, which is the score of a possible sum(ai)=1.0.

Therefore, the sedative score is (0.5/1)*100=50% percent sedative in the secondary terpenes. That is, 50% of the secondary terpene abundance totals are sedative.

The scoring could alternately be scaled as a percent of the total terpene contents (instead of sum of secondary terpene contents, where ai is all the terpene abundances, primary and secondary are summed up.

Adding a Primary Terpene to the Scoring in the Secondary Clade Evaluation of Therapeutic Effects:

For example, alpha pinene is the only primary terpene that is an AChE (acetylcholinesterase) inhibitor (AChEI). Therefore, it may be desirable to include alpha pinene with the secondary terpenes in the scoring computation for cognitive support with all other AChEI's.

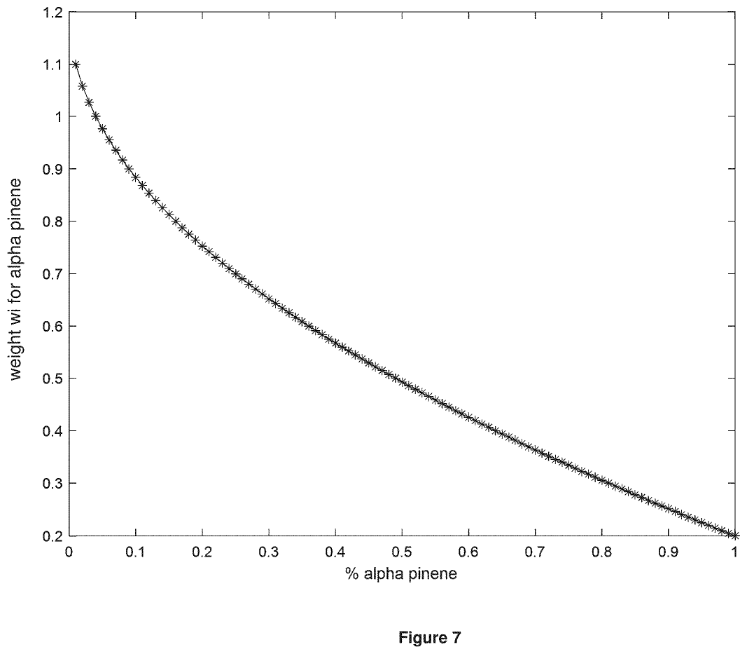

Alpha pinene can be expected to have a decaying weighting factor profile relative to concentration because the enzyme inhibitor activity is not linear with concentration, as shown in FIG. 7.

Because enzyme inhibition is not proportional to concentration, we can use a 0.5 weight for low alpha pinene (<0.5%) and a 0.2 weight or proportionally smaller weight for high alpha pinene, say, >0.5%

Dscore including a pinene=[(1−sqrt(a)+0.2)*(aₐₚᵢₙₑₙₑ)²+(a₂)²+(a₃)²+ . . . (aₙ)²]

Therefore, in this case, the weight is (1−(a)+0.2).

Flow Charts Depicting Analyses

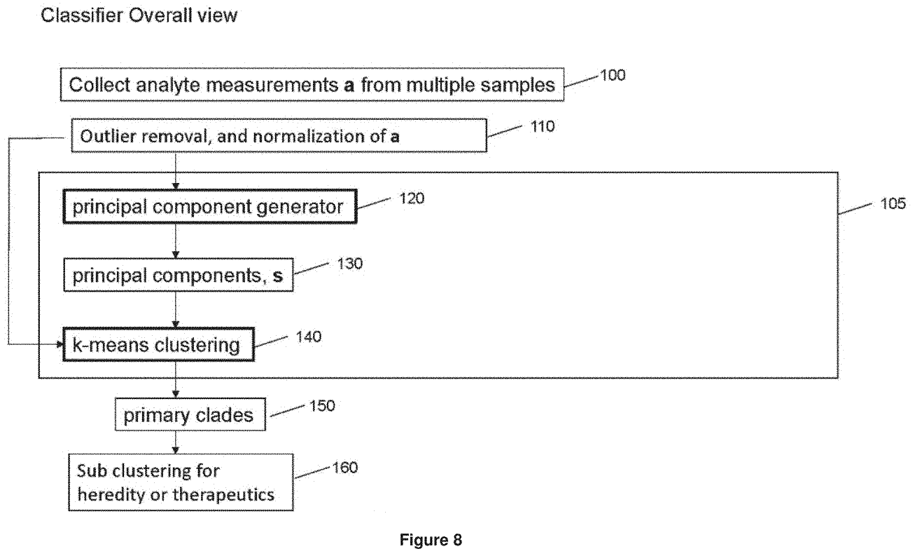

FIG. 8 is a flow chart depicting an example of the overall classification scheme obtained by the methods provided herein

In FIG. 8:

100—collect analyte vector a for sample(s)

110—outlier removal and normalization a=a/|a|₂ where |a|₂ is the second norm

120—principal component decomposition of a

130—scores, s from principal components

140—Kmeans clustering on either a or PCA scores s

150—assign primary clade membership

160—optionally, assign secondary clade clustering membership

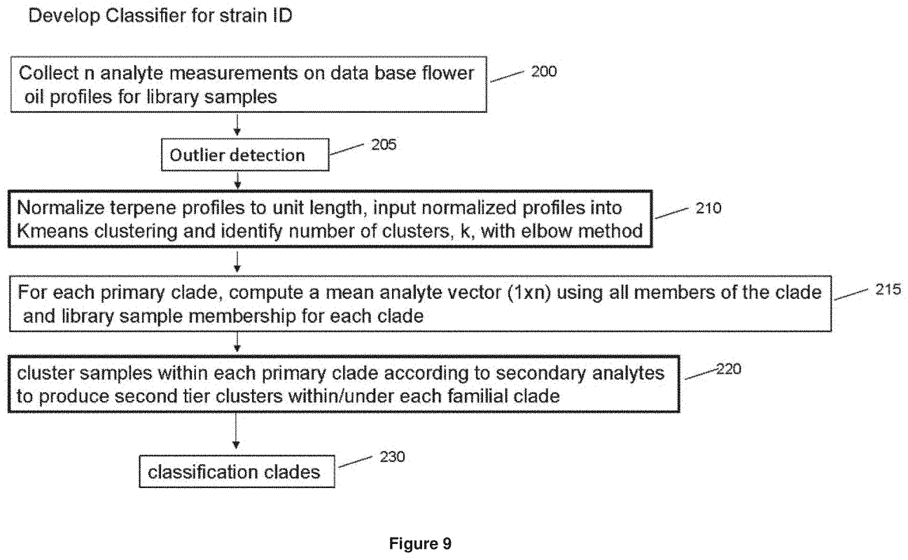

FIG. 9 is a flow chart depicting an example of how the classification clades are obtained.

In FIG. 9:

200—Collect a library (e.g., several hundred to thousand or more) samples each, with n analyte abundance (%) measurements, a, on data base flower oil profiles for library samples

205—if desired, perfom outlier screening that includes one or more of the following: screen sample data for total terpene content above a threshold percent of dry weight of the sample (e.g., >1%), remove aged samples (e.g., less than 10% decarboxylation to give THC), screen for known synthesis co-products that should either be in known ratios or co-abundances (occur together). For example, if the analysis certificate (COA) does not have the known co-abundances or acceptable ratios of beta caryophyllene and humulene and alpha and beta pinene, remove from library. Other known ratios are between terpinolene and: alpha phellandrene, 3 careen and alpha terpinene, gamma terpinene as described elsewhere herein.

210—Normalize terpene profiles to unit length, a=a/|a|₂ where |a|₂ is the second norm, then input the normalized profiles into Kmeans clustering and identify number of clusters, k, using the elbow method

215—Average all members of each clade over each analyte this vector of averages is the clade centroid a_{cj} where Centroid a_{cj}=[a₁ⱼ a₂ⱼ a₃ⱼ . . . aₙⱼ] for each of j centroids

220—Cluster analyte data within each clade, find tier 2 groupings (secondary clades using secondary terpenes or secondary terpenes and weighted primary terpenes) of analyte similarity.

230—clade classification system ID includes primary clade and secondary clade cluster numbers

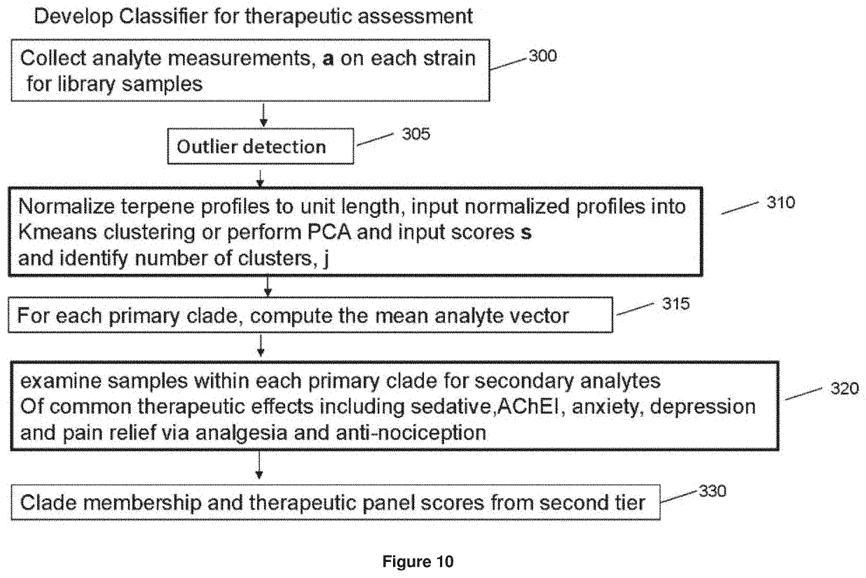

FIG. 10 is a flow chart that is a specific example of the flow chart depicted in FIG. 9, where the secondary clades are clustered within the primary clades according to therapeutic activity.

In FIG. 10:

300—Collect a library (e.g., several hundred to thousand or more) samples each, with n analyte abundance (%) measurements, a, on data base flower oil profiles for library samples

305—if desired, perfom outlier screening that includes one or more of the following: screen sample data for total terpene content above a threshold percent of dry weight of the sample (e.g., >1%), remove aged samples (e.g., less than 10% decarboxylation to give THC), screen for known synthesis co-products that should either be in known ratios or co-abundances (occur together). For example, if the analysis certificate (COA) does not have the known co-abundances or acceptable ratios of beta caryophyllene and humulene and alpha and beta pinene, remove from library. Other known ratios are between terpinolene and: alpha phellandrene, 3 careen and alpha terpinene, gamma terpinene as described elsewhere herein.

310—Normalize terpene profiles to unit length, a=a/|a|₂ where |a|₂ is the second norm, then input the normalized profiles into Kmeans clustering and identify number of clusters, k, using the elbow method

315—Average all members of each clade over each analyte this vector of averages is the clade centroid a_{cj} where Centroid a_{cj}32 [a₁ⱼ a₂ⱼ a₃ⱼ . . . aₙⱼ], ad for each of j centroids

320—Cluster analyte data within each clade, find tier 2 groupings (secondary clades using secondary terpenes or secondary terpenes and weighted primary terpenes) of analyte similarity.

330—clade classification system ID includes primary clade and secondary clade cluster numbers

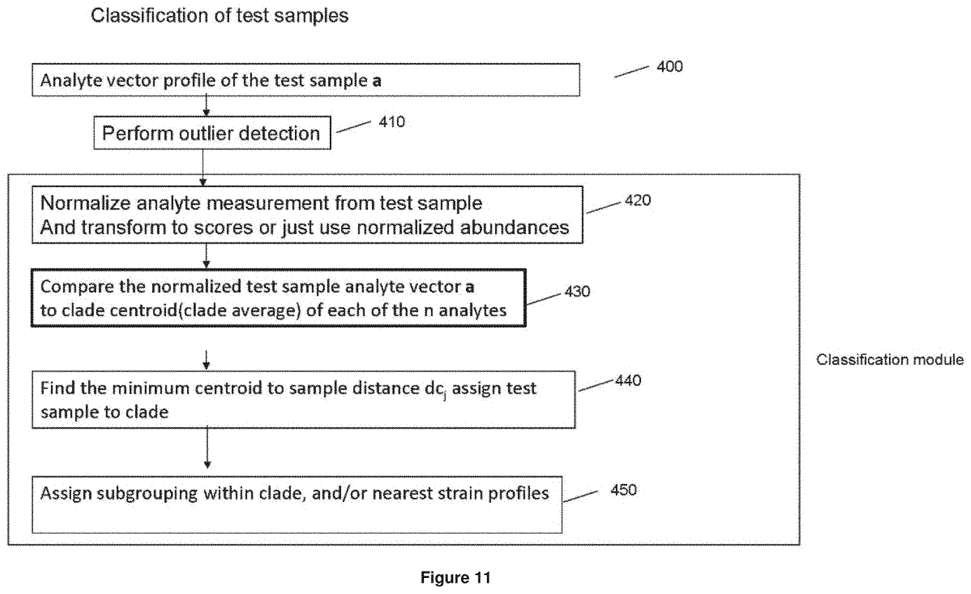

FIG. 11 is a flow chart that depicts an example of how to classify a test sample based on the clades that have been constructed from a library

In FIG. 11:

400—collect a the 1×n sample analyte vector of analytes

410—Perform outlier detection for known co synthesis products and ageing ratio of THCA:THC, and screen Screen THC/THCA ratio as less than 0.1 (10%)

420—Normalize sample analyte vector, a=a/|a|₂ where |a|₂ is the second norm, then either use directly or perform a PCA to get scores, s.

430—measure distances dcⱼ to each clade centroid a_{cj}=[a₁ⱼ a₂ⱼ a₃ⱼ . . . aₙⱼ] for each of j centroids
dcⱼ=[w₁(a₁−a_{c1j})²+w₂(a₂− a_{c2j})²+w₃(a₃−a_{c3j})²+ . . . wₙ(aₙ−a_{cnj})²]^{1/2} for weighting
dcⱼ=[(a₁−a_{c1j})²+(a₂−a_{c2j})+(a₃−a_{c3j})²+ . . . (aₙ−a_{cnj})²]^{1/2} no weighting of analytes
where i is analyte number j is clade number
If using scores substitute s for a

440—Find minimum distance dcj, assign test sample to clade

450—Subcluster within clade, calculate distances, dcⱼ, to of sample to subcluster centers and assign sub clade grouping

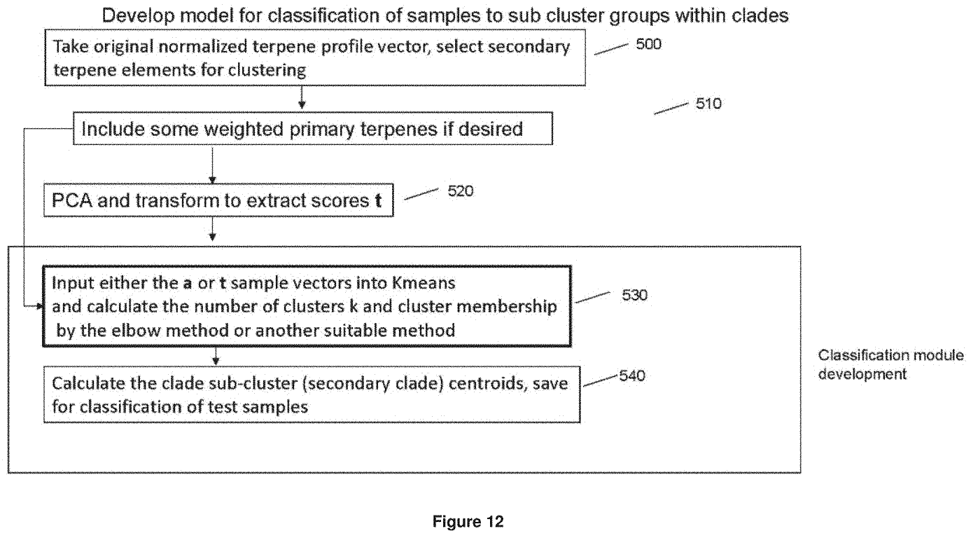

FIG. 12 is a flow chart that depicts an example of an overview of how to sub cluster terpenes within the primary clades (i.e., obtain secondary clades)

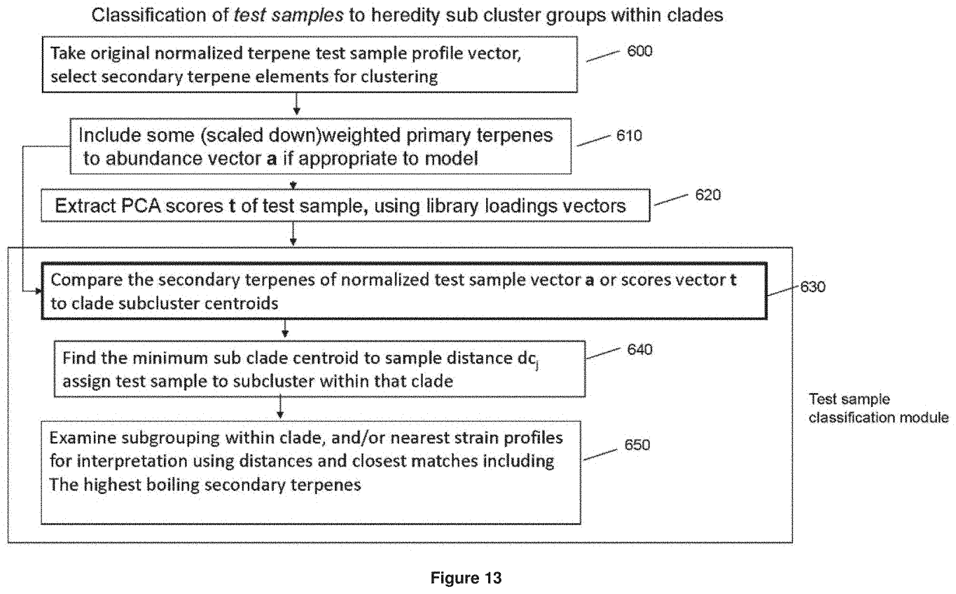

FIG. 13 is a flow chart that depicts an example of how to assign test samples to secondary clades that are scored for heredity.

The analysis depicted in FIG. 13 uses the same Kmeans process as for construction of the primary clades, except, in embodiments no further normalization of the secondary terpenes is needed. Subsets of the secondary terpenes that are present as high boilers (ratios that are consistent over time due to minimal dissipation or other losses) can be used to get a more accurate final match. For example, after secondary clade assignment, a reduced set of high boiling terpenes present in a test sample from a target strain can be used as a final fingerprint to compare against member strains of a secondary clade. Some of these persistent secondary terpenes are alpha bisabolol, alpha terpineol, Guiaol, nerolidol, fenchol, and linalool. This reduced set vector should be most consistent over time. This approach can be useful when looking at the small amounts of these terpenes after filtering out the more abundant primary terpenes in the primary clade classification.

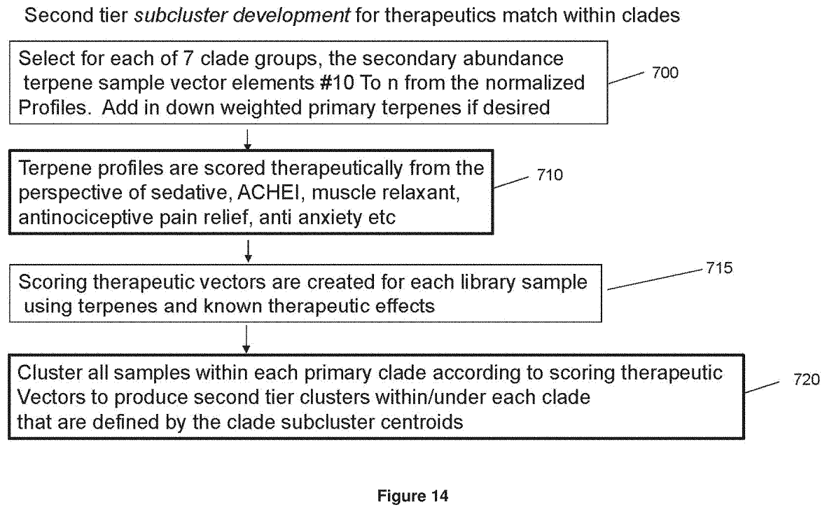

FIG. 14 is a flow chart that depicts an example of an overview of how to construct secondary clades based on therapeutic activity

Scoring can be defined by %, that is the percent of secondary terpenes that are sedative in action, percent that are anti-anxiety, percent that offer ACHEI for memory and cognitive support, percent that offer antinociceptive pain relief, etc. The scores of more than therapeutic effect can be combined to give a combined acore. Alternately, the therapeutic effects can also be scored individually, for example, the % sedative content of secondary terpenes can be used to select a sedative strain for insomnia

The scoring vectors are useful for clustering (secondary clades) to match therapeutic effects of strains within the primary clade. Therapeutic scoring can also be used to obtain clades based on gender profiling, e.g., when one gender responds better to treatment with a terpene or set of terpenes than the other gender.

The therapeutic scoring vector is represented as ts=[ts1 ts2 ts3 . . . tsn] for n therapeutics this vector is potentially sex dependent and it can be used to generate sex dependent Kmeans groups within secondary clade sub-clusters (tertiary clades, e.g.) for gender specific therapeutic effects. In embodiments, therapeutic scoring can be weighted to reflect potenc, e.g., when dose response information is available. In certain embodiments, PCA can mask the interpretation of the overall therapeutic activities in a secondary clade and is not used in clustering the therapeutics into secondary clades.

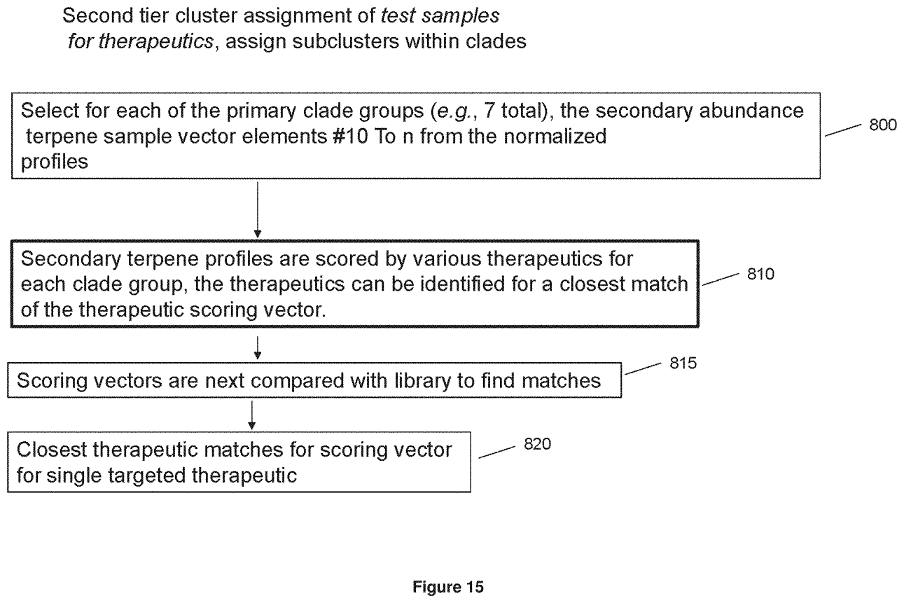

FIG. 15 is a flow chart that depicts an example of how to assign test samples to secondary clades that are scored for therapeutic activity.

For each test sample, individual therapeutic effects are scored and the combined therapeutic effect or subset thereof is matched to clades constructed from the reference library strains. Therapeutics of primary terpenes can be added in the model but generally are weighted down, e.g., based on potency, to prevent domination of the overall therapeutic representation.

Use of Devices and Programs

The classification systems and methods provided herein can include the use of a machine containing one or more microprocessors and memory, which memory includes instructions executable by the one or more microprocessors and which instructions executable by the one or more microprocessors are configured to (A) access the measured amounts of one or more individual analytes from a plant sample, and a measured amount of the total analytes in the plant sample, wherein the analytes belong to the same chemical class; (B) for each plant sample, based on the measured amounts in (A): (i) determine the abundance of the one or more individual analytes in the sample relative to the total amount of analytes in the sample, thereby obtaining the relative abundance of the one or more individual analytes in the sample, (ii) determine the order of relative abundance, from highest to lowest relative abundance or from lowest to highest relative abundance, of the one or more individual analytes in the sample, and (iii) based on (i) and (ii), determine an abundance profile of the analytes for each plant sample; (C) optionally, for each plant sample, determine whether the sample is an outlier and, if the plant sample is an outlier, not subject the sample to (D) and (E) or, determine the difference between the original analyte abundance profile of the sample and the analyte abundance profile that renders the sample an outlier and, based on the difference, reconstruct the original analyte abundance profile of the sample before subjecting the sample to (D) and (E); (D) for each plant sample not identified as an outlier or, if an outlier, reconstructed to its original analyte abundance profile, normalize the measured amounts of the one or more individual analytes, thereby obtaining, for each plant sample, a normalized abundance profile containing normalized analyte levels of the one or more individual analytes; and (E) based on the normalized abundance profiles of the analytes for each plant sample, assign plant samples comprising the same normalized abundance profiles to a group, wherein each group is a primary clade that comprises plant samples comprising the same chemotype. In embodiments, the instructions executable by the one or more microprocessors can further be configured to (1) for each plant sample in at least one primary clade, obtain the identity and/or normalized measured amount of (i) one or more additional analytes that are different from the analytes measured to assign the primary clade, or (ii) a mixture of one or more individual analytes measured to assign the primary clade and one or more additional analytes that are different from the analytes measured to assign the primary clade, wherein the additional analytes are associated with heredity and/or a known therapeutic effect; (2) for each plant sample, based on the identity and/or normalized measured amount of amount of (i) or (ii), obtain one or more profiles selected from among a heredity profile of analytes and a therapeutic profile of the analytes of (i) or (ii); and (3) identify plant samples within each primary clade that contain the same heredity profiles and/or therapeutic profiles, as belonging to the same secondary clade. In embodiments, the analytes are terpenes and in certain embodiments, the plant samples are from Cannabis plant strains.

Also provided herein is a non-transitory computer-readable storage medium with an executable program stored thereon, where the program instructs a microprocessor to perform the following: (A) access the measured amounts of one or more individual analytes from a plant sample, and a measured amount of the total analytes in the plant sample, wherein the analytes belong to the same chemical class; (B) for each plant sample, based on the measured amounts in (A): (i) determine the abundance of the one or more individual analytes in the sample relative to the total amount of analytes in the sample, thereby obtaining the relative abundance of the one or more individual analytes in the sample, (ii) determine the order of relative abundance, from highest to lowest relative abundance or from lowest to highest relative abundance, of the one or more individual analytes in the sample, and (iii) based on (i) and (ii), determine an abundance profile of the analytes for each plant sample; (C) optionally, for each plant sample, determine whether the sample is an outlier and, if the plant sample is an outlier, not subject the sample to (D) and (E) or, determine the difference between the original analyte abundance profile of the sample and the analyte abundance profile that renders the sample an outlier and, based on the difference, reconstruct the original analyte abundance profile of the sample before subjecting the sample to (D) and (E); (D) for each plant sample not identified as an outlier or, if an outlier, reconstructed to its original analyte abundance profile, normalize the measured amounts of the one or more individual analytes, thereby obtaining, for each plant sample, a normalized abundance profile containing normalized analyte levels of the one or more individual analytes; and (E) based on the normalized abundance profiles of the analytes for each plant sample, assign plant samples comprising the same normalized abundance profiles to a group, wherein each group is a primary clade that comprises plant samples comprising the same chemotype. In embodiments, the program can further instruct the microprocessor to perform the following: (1) for each plant sample in at least one primary clade, obtain the identity and/or normalized measured amount of (i) one or more additional analytes that are different from the analytes measured to assign the primary clade, or (ii) a mixture of one or more individual analytes measured to assign the primary clade and one or more additional analytes that are different from the analytes measured to assign the primary clade, wherein the additional analytes are associated with heredity and/or a known therapeutic effect; (2) for each plant sample, based on the identity and/or normalized measured amount of amount of (i) or (ii), obtain one or more profiles selected from among a heredity profile of analytes and a therapeutic profile of the analytes of (i) or (ii); and (3) identify plant samples within each primary clade that contain the same heredity profiles and/or therapeutic profiles, as belonging to the same secondary clade. In embodiments, the analytes are terpenes and in certain embodiments, the plant samples are from Cannabis plant strains.

Generating a classification system using the one or microprocessors, or assigning a sample from a plant strain to a primary clade and, optionally, one or more secondary clades, can involve one or more, or several manipulations of the abundance, heredity and/or therapeutic profiles, which can require the use of one or more or multiple computers. A report can be generated by a computer or by human data entry, and can be communicated in person or by electronic means (e.g., over the internet, via computer, via fax, from one network location to another location at the same or different physical sites), or by other method of sending or receiving data (e.g., mail service, courier service and the like). The report can include information regarding whether one or more plant strains have the desired characteristics requested by a customer for, e.g., breeding, cultivation as a crop, or therapeutic use. The outcome can be transmitted to a customer, such as a plant breeder, farmer, health care professional or subject/patient in need of treatment with one or more plant strains/portions thereof/products thereof/extracts thereof, in a suitable medium, including, without limitation, in verbal, document, or file form including, but not limited to, an auditory file, a computer readable file, a paper file, a laboratory file or a medical record file.

Methods of Use

The classification methods and systems provided herein can be used to identify plant strains having a desired phenotype for a variety of uses, e.g., for breeding, for cultivating a crop, or for medicinal use. For example, for breeding or for cultivating/growing a crop, plant strains having primary and/or secondary analyte profiles that have certain ancestry/heredity, or that renders them resistant to or suitable for growth under certain environmental conditions or in certain geographic locations, can be selected and the selected plant strains can be bred or cultivated. For a therapeutic application, for example, a subject can be treated with a plant strain that has a therapeutic profile of interest based on the scoring of therapeutic factors such as, for example, gender selective effects, sedation, anxiety, and the like. The methods, e.g., of breeding, cultivation and treatment provided herein can be based on the consistent selection of plant strains according to the desired phenotype/chemotype, for example, when the relationship between the genotypes and the phenotypes/chemotypes of the plant strains are not well established.

Products

Test samples analyzed by the methods provided herein can be assigned to primary clades and, optionally, one or more secondary clades. The test samples or their corresponding plant strains or portions thereof can then be packaged, or processed as needed and then packaged, into different products depending on their use, and the packaged products can be labeled, e.g., in color codes or words or bar codes, based on the phenotype(s) that they are selected for. For example, if the application is in agriculture (e.g., for breeding or planting), then in embodiments, seeds or whole plants can be selected based on the desired breeding and/or heredity and/or therapeutic activity and/or resistance to or favoring an environmental condition or a geographic location and the like, by reading color coded labels or words or bar codes. If the application is in therapeutics, products such as edibles, inhalables and topicals used for therapeutic benefit can be selected based on the desired therapeutic effects by reading color coded labels or bar codes. In embodiments, the samples can be labeled in color codes. For example, if the test sample is limonene dominant, then a color, e.g., yellow, can be assigned to the limonene dominant primary clade and the test sample or other corresponding product can be labelled yellow. If the test sample additionally was assigned heredity, therapeutic, or other characteristics based on secondary clade analysis, secondary colors can be added to the label, e.g., as rims around the “primary” color code or as rays originating from the center of the primary color code. For example, if the test sample assigned to the limonene dominant primary clade additionally has sedative properties, a color can be assigned to sedation, e.g., blue and be represented as a rim around the yellow “primary” color. Additional colors can be added to the labels as appropriate e.g., a color can be assigned to test samples that have a high content of women-specific therapeutic terpenes, or brain wave influencing terpenes. Such a labeling scheme can permit comprehensive visualization of the phenotype of a test sample in a simplified manner. Thus, also provided herein are products that are labelled according to their classification obtained by the methods provided herein, and articles of manufacture that include such products. In embodiments, the articles of manufacture can be used in methods of industrial, agricultural or medicinal use such as, for example, in breeding, in cultivating/growing crops, or in methods of treatment.

### EXAMPLES

The examples set forth below illustrate certain embodiments and do not limit the technology. Certain examples set forth below utilize statistical methods as described herein and as known in the art.

### Example 1: Generation of Clades Based on Terpene Profiles

A. Sample Collection and Preparation

Cannabis flower samples (1683 total) were obtained from customers or were collected from growers who volunteered samples. Each of the flower samples was homogenized with an herb shredder and weighed into a 15 ml centrifuge sample tube to a nominal weight of 0.5 g+/−0.050 g. 10 ml of acetone was added to the sample, followed by 15 minutes in an ultrasound bath, 1 minute of vortexing and then 15 minutes of sonication to fully extract the sample. Samples were then diluted 50× in methanol/water and run on a Shimadzu Gas chromatograph/quadrupole mass spectrometer. Stock calibration solutions were prepared for 43 terpenes and a calibration was developed and applied to all sample data to generate each sample terpene concentration profile.

To confirm peak identification, selected samples were analyzed by GC-MS using a single quadrupole MS-detector. Compounds were compared based on their mass spectra and retention, and the NIST library was used to assist in compound identification (Standard Reference Data Program of the National Institute of Standards and Technology, as distributed by Agilent Technologies). For quantitative analysis, peak area values were quantified (in mg/g of plant material) with the use of calibration curves. Monoterpenes and sesquiterpenes were quantified using the calibrated standards. Each calibration curve consisted of five different concentration levels in the range of 0.005-0.1 mg/mL. Calibration curves were regularly prepared throughout the duration of the study. The resulting quantitative data were not corrected for residual moisture content of the samples.

Multivariate data analysis was conducted using Matlab 2015b software with the statistics and machine learning toolbox. Hierarchical clustering with PCA inputs was used to explore structure in the terpene data set initially and get an estimate for the number of dusters to test in KMeans clustering, which was then used to define the clade membership.

Terpene concentration profile data from 1683 cannabis samples were separated according to strain names, and from among the replicate named samples a search for different chemotypes was undertaken. Different chemotypes within a strain name were defined as a change in at least one of the top 6 most abundant terpenes. For example, if myrcene was most abundant in one plant sample by 10-20% of value and then was second most abundant in a second plant sample by 10-% of value, it would trigger a chemotype change for the second sample. Different chemotypes within the same strain name were included in the library for up to 2 phenotypes per strain name. Measured samples that appeared to replicate the terpene concentration profile were excluded; among replicates, the exemplar with the highest total terpene content was retained. A total of 375 strain phenotypes was analyzed for classification into clades.

B. Analysis of the Samples

These terpene concentration profiles contain the concentrations of 43 terpenes (measured against the standards, as described above), making a 1×43 vector a defining the 43 terpene concentrations found in each sample, aᵢ. This vector of 43 terpene concentrations is defined as the strain “terpene profile” or strain chemovar profile.

Outlier Identification

Each sample terpene vector was subjected to a series of outlier tests to ensure adequate data quality. Outlier tests are designed to use ageing and the known co-production of terpenes to exclude the sample profiles that do not conform to the expected genetic co-production of terpenes by TPS (terpene synthase) enzymes. Reasons for failure to conform can include errors in COA (Certificate of Analysis) and excessive ageing or sample handling losses of terpenes. For example, some terpenes (e.g., monoterpenes) can be lost during processing due to their low boiling point or high surface area. The outlier tests can be one or more of the following:
1) The percentage of decarboxylated tetrahydrocannabinolic acid (THCA) in the sample. Decarboxylated THCA is tetrahydrocannabinol (THC), which is the psychoactive form. The percentage of THC is obtained using the equation: ([THC]/[THCA+THC])×100, where [THC] is the concentration of THC and [THC+THCA] is the total concentration of THC and THCA in the sample. If the THC percentage is greater than 10%, the sample is excluded from the data base due to sample storage, ageing or handling issues which can cause depletion of terpenes.
2) The beta caryophyllene/humulene ratio produced by TPS (terpene synthase) genes has averaged 3.2:1 but a range of 2:1 to 6:1 is acceptable due to analytical error and storage/handling losses and the rest are screened out as outliers.
3) If alpha pinene is greater than 2× the limit of quantization, beta pinene must be detected or the sample is declared an outlier as these are co-produced by the TPS genes, with alpha pinene/beta pinene ratios from 0.3:1 to 6:1.
4) If beta pinene is at limit of quantitation (LOQ), alpha pinene must be detected or the sample is identified as an outlier.

Other tests for identifying outliers can include: terpinolene/3-carene ratios at 15:1, with a range from 10:1 to 38:1, terpinolene/alpha phellandrene ratios at 16:1, with a range from 5:1 to 30:1, terpinolene/alpha pinene ratios from 20:1 to 100:1, alpha terpineol/fenchol ratios from 0.3:1 to 2.5:1, terpinolene/gamma terpinene ratios at 50:1, with a range from 20:1 to 120:1 (most of the abundance data is near the limit of detection (LOD), making the range of ratios broader), and terpinolene/sabinene or sabinene hydrate ratio of about 100:1. In addition, samples with <0.9% total measured terpenes (based on inflorescence dry weight) were excluded as outliers from both the library and the strain matching of test samples.

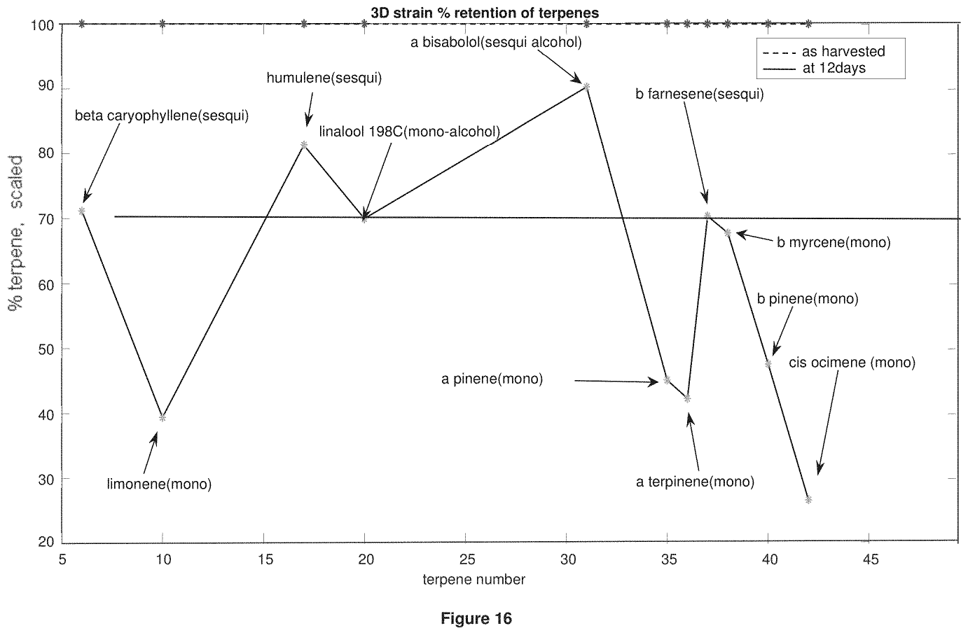

In FIG. 16, the percent residual terpenes from day after harvest to a 12-day uncontrolled environment shows an approximate dissipation in order of the expected volatility in an accelerated ageing/storage experiment.

The observed order of persistence was found to be sesquiterpene alcohol>sesquiterpene>mono terpene alcohol>mono terpene. This order correlated with the molecular weights of the terpenes and the presence/absence of alcohol functional groups which are known to lower volatility via hydrogen bonding. The greatest storage dissipation observed was for mono terpenes at high abundance, as theft dissipation rate is influenced not only by boiling point but also concentration gradients that drive the rate of diffusion within the flower oils/structure by Ficks laws of diffusion. Weighting schemes as provided herein and as known to those of skill in the art can be used to limit the impact of storage and handling on terpene chemovar or ancestry identification and to predict sample storage and ageing impact on therapeutic effects. Alternately, the less abundant terpenes can be analyzed separately from the more abundant volatile primary terpenes. For example, if the therapeutic target is antinociceptive pain relief, the powerful antinociceptive pain relievers of trans nerolidol, alpha phellandrene, alpha terpineol, and alpha bisabolol are going to have more impact than the dissipation of primary (more abundant, higher dissipating rate) terpenes like myrcene and limonene in storage.

Terpene Quantification

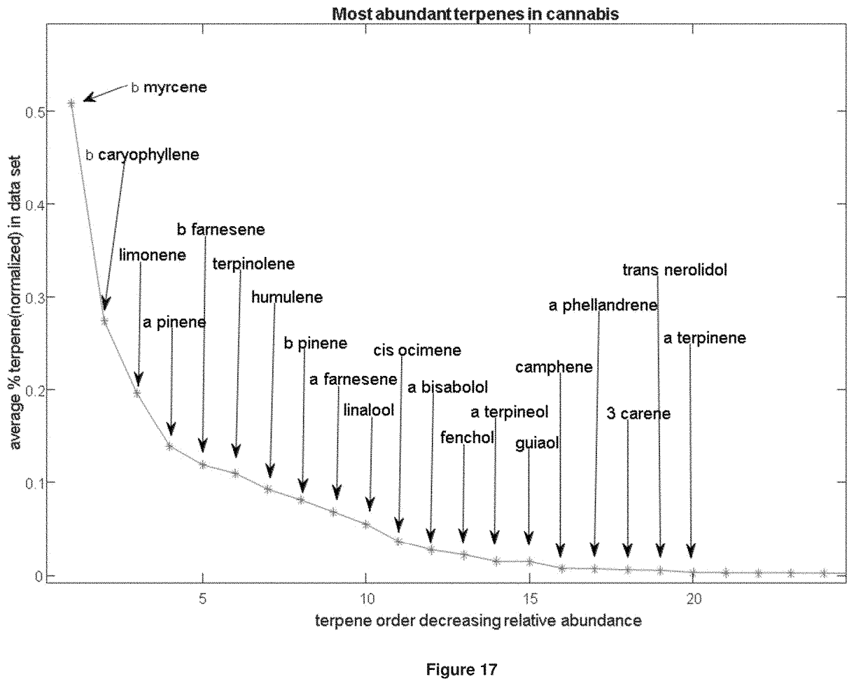

The average relative abundance/levels in % of terpenes observed in the 375 strain phenotypes analyzed is presented in FIG. 17. It was observed that about 20 of the most abundant terpenes were present at non trace levels, representing measured averages at well above detection limits. The order of relative abundance was similar to that found in some other studies, with the exception that beta farnesene, which most other strain databases did not include in theft terpene analysis, was identified as the 5^{th} most abundant terpene in the library samples collected, based on the average concentration of terpenes over all identified phenotypes. As seen in FIG. 17, there is an order of magnitude range in average relative abundance among even the most abundant (primary) terpenes.

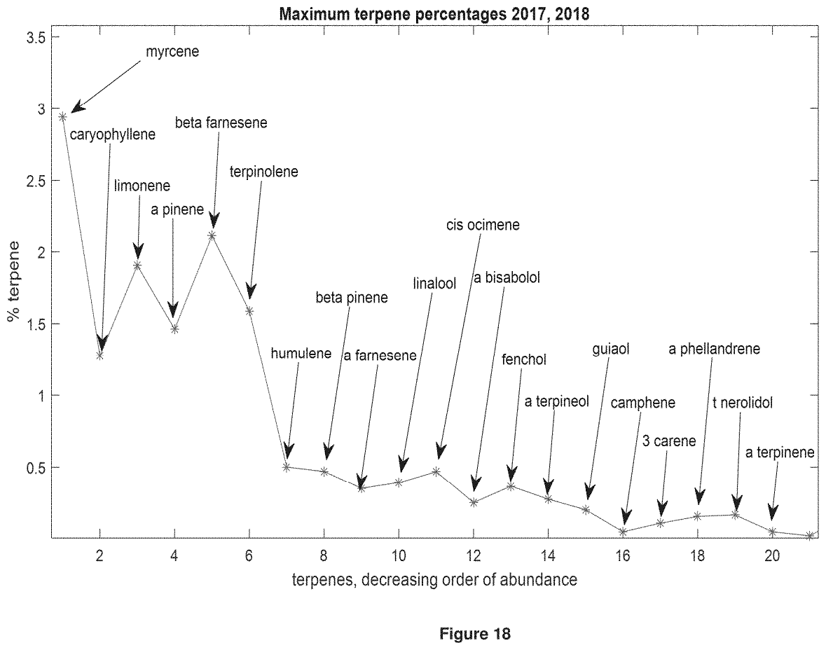

In FIG. 18, the maximum concentrations observed for each terpene is presented. The results showed that terpenes 1-6 (from left to right) were the dominant terpenes in the Cannabis strains sampled. The dominant terpenes were up to 5-6 times higher than all other terpenes in Cannabis. Because it is likely that humulene (alpha caryophyllene), beta pinene, and alpha farnesene are co-products of terpene synthase reactions that make beta caryophyllene, alpha pinene, and beta farnesene in the plant, these three terpenes were included in the classification of the primary clades (most abundant terpenes) making it a primary set of 9 terpenes. Correlations in the data between these isomers support that they may be produced by the same terpene synthase enzymes as their closest constitutional isomers and that they are not independent in abundance.

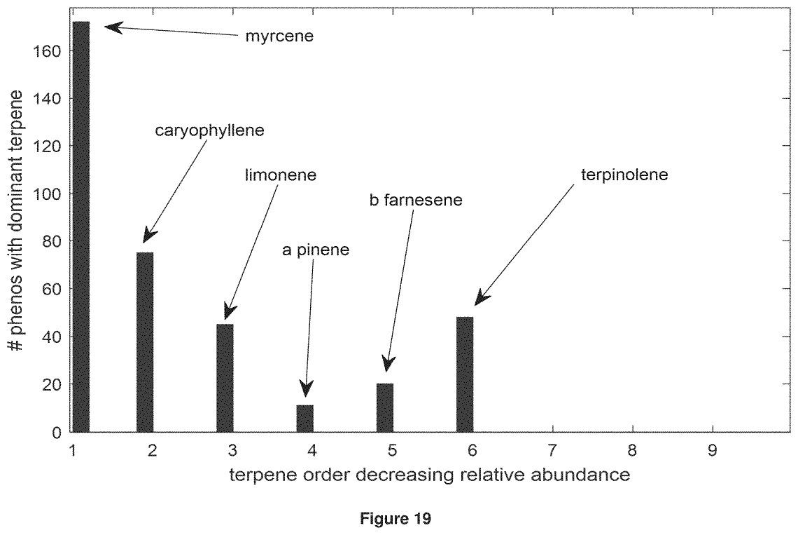

The top 10 most abundant terpenes measured included, in order: beta myrcene, beta caryophyllene, limonene, alpha pinene, beta farnesene, terpinolene, humulene, beta pinene, alpha farnesene, and linalool. Of these top 10 most abundant terpenes, 6 were measured as the most abundant terpene in any one strain. The distribution of dominant terpenes in this data set is presented in FIG. 19.

It can be seen from FIG. 19 that beta myrcene is the most abundant terpene in about half the strain data base. Six terpenes were observed to be most abundant in at least ten strains each. No other terpene was most abundant in any strain phenotype. These six terpenes also have 3 isomers that are believed to be connected through synthesis pathways, as described above. Therefore, this first group of 9 terpenes were identified as “primary” terpenes that were classified into “primary” clades, based on relative abundance. The “secondary” terpenes are defined here as the 10^{th} to 20^{th} most abundant terpenes depicted in FIG. 2 above which, although on average are approximately an order of magnitude lower in abundance than the primary terpenes, can be considerably potent because medical and bioactivity effects at fixed dosage also can vary by at least an order of magnitude. The secondary terpenes are subjected to cluster analysis (with or without some of the primary terpenes, which can be weighted based on their relative potency) within each primary terpene group according to ancestry/lineage, therapeutic effects and other agricultural, industrial or medical applications.

Terpene Classification

Multivariate data analysis was conducted using Matlab 2015b software with the statistics and machine learning toolbox. Hierarchical clustering with principal component analysis (RCA) inputs was used to explore structure in the primary terpene data set initially and get an estimate for the number of clusters to test in KMeans clustering, which was then used to define the clade membership. The library data were refined to one terpene profile per strain phenotype and examined for content. A Hierarchical Clustering Analysis, HCA, was used to visualize the high dimensional sample clustering structure of terpene strain profiles using k means distances. Preprocessing of terpene profiles included scaling the overall profile vector by its second norm. The RCA scores were then used as inputs to the hierarchical clustering analysis, HCA, using k means distances in Matlab 2015b software. The resulting dendrogram was suggestive of at least 7 major clusters, each of which is termed a clade. Details regarding the statistical methods are as known to those of skill in the art and as described elsewhere herein.

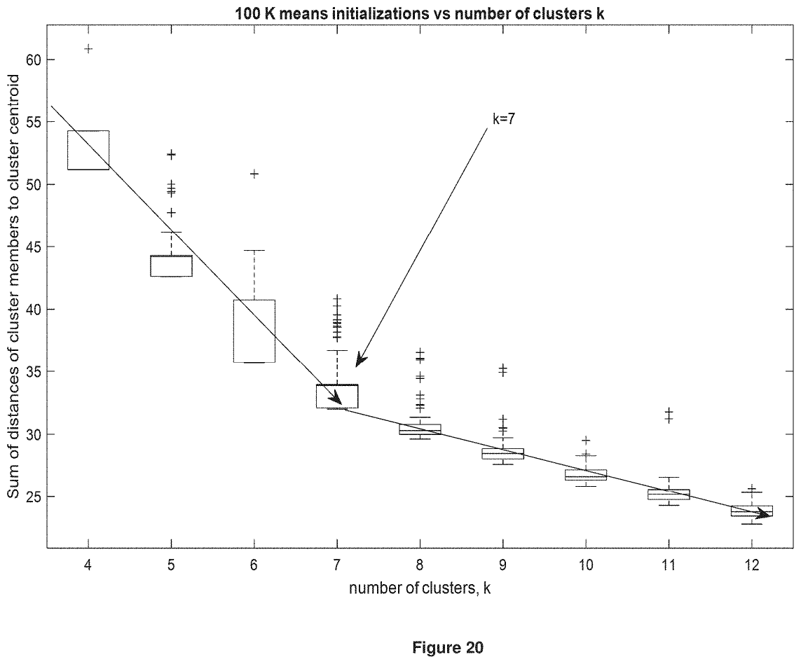

After hierarchical clustering, the number of clade clusters, k, was selected based on the “elbow point” of the KMeans within cluster distances for k from 4 to 10. In this first tier of clustering (primary terpenes classified into clades), a Euclidean distance metric (Equation 1; see section on Statistical Analysis) was used. The results are shown in FIG. 20.

With all the genetic crosses in the data base, the clustering data structure might be expected to be closer to a continuum rather than clear clustering structures. Cluster selection using the elbow method often can be ambiguous due to deviations from normality in clustering data. The results above however show that the determination of k was clear at k=7, as the inflection was obvious. After k=7, reduction in the total cluster distances tapered off to a gradual, constant decline. As future data is collected, more complexity can be uncovered in the data structure. For example, new strains that are highly dissimilar in their chemistry profile compared to existing database strain samples can entail the use of additional clade groups in the first tier. In addition, new terpenes can be added to the strain profiles, to more completely understand the whole range of Cannabis strain offerings. Assigning future strains to the clades in the first tier is performed by a nearest Euclidean distance to each centroid as described herein (see, e.g., Equation 1 in the Statistical Analysis section). The distance was computed for all 7 centroids and the smallest distance determined clade membership of the new strain. Distances to other clades and the n nearest strains also can be a potentially useful secondary metric for use in therapeutic assessment. Implementation of distance weighting using Equation 2 (see Statistical Analysis section) can enhance a more focused therapeutic, heredity, agricultural or other property-based second or more tier classifier (i.e., secondary, tertiary or other higher order clades), depending on the known information about these properties. Alternately, the information can be excluded or if the information is absent, all information weights are all set to 1 or 0 in the second tier clustering.

Primary Clades

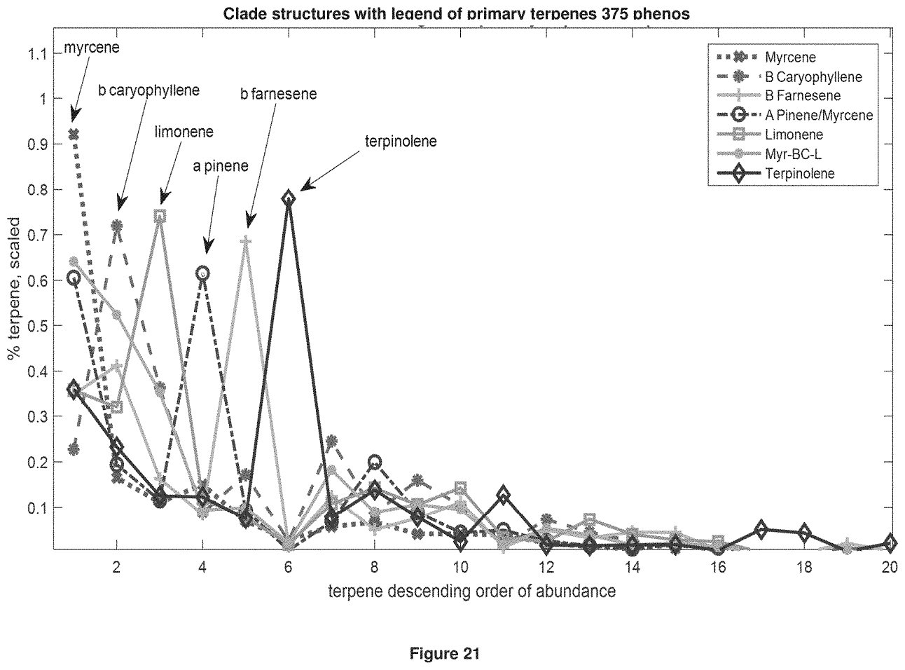

The 7 clade terpene centroids obtained by analyzing the 9 primary terpenes as described above are presented in FIG. 21. Of the six most abundant of the primary terpenes, it was found that all were represented as most abundant or co-most abundant in the clade centroids. The 7 primary clades identified are as follows:

Clade 1: Alpha pinene and myrcene co-dominant. These terpenes are known for anti-anxiety, enhanced cerebral function, anti-hypertensive effects (alpha pinene) and some analgesic pain relief (myrcene).

Clade 2: Limonene dominant, with beta caryophyllene and myrcene as the next most abundant, L-BC/M. This group has sedative and anti-anxiety effects, with body relaxation and pain relief.

Clade 3: Co-dominant beta caryophyllene and limonene, with myrcene at lower abundance, designated as BC/L-M. The group has anti-anxiety, pain relief, anti-depression and moderate sedative effects.

Clade 4: Myrcene dominant for some moderate analgesic pain relief but the effects of the other primary terpenes in this clade (at low abundance) are variable and include, for example, cognitive function and memory support, sedation, mental focus and relaxation. There are potentially 3 therapeutic groups within this clade.

Clade 5: Beta farnesene dominant, this group produces relaxation, moderate sedation, good mental clarity.

Clade 6: Terpinolene dominant, most of this clade is activity supporting with some muscle relaxation but mostly no sedation. This clade is mental energy and creativity enhancing, with a relaxed focus for morning or evening use.

Clade 7: Myrcene, beta caryophyllene, limonene, designated as M-BC-L. This clade can provide the effects of anti-anxiety, anti-depressant, variable sedation, relaxation and body pain relief.

Secondary Clades

The secondary terpenes (10^{th} to 20^{th} most abundant) can be used for clustering within the primary clades, with or without weighting factors based on known effects and with or without adding primary terpenes to the analysis (with weighting factors where appropriate), to fine tune the classification of strains based on properties other than terpene abundance, such as ancestry/heredity, therapeutic effects or characteristics useful in agriculture, such as plant strains favored for growth under certain conditions. Kmeans clustering is used to divide the first tier of clades, and in the second tier it is used to cluster within clades.

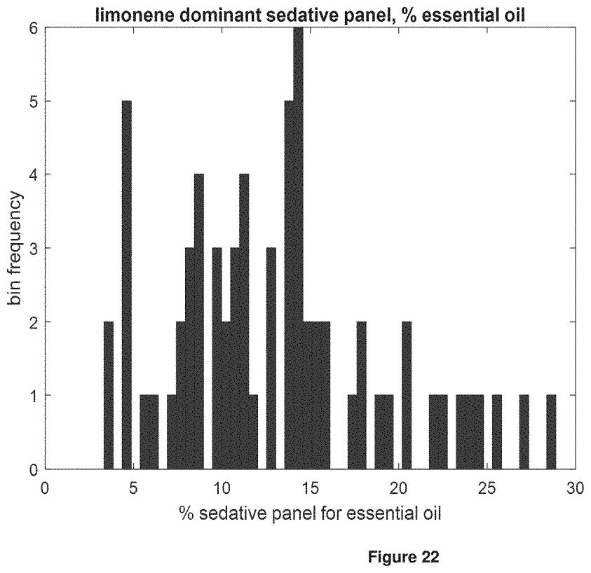

For example, in FIG. 22, the limonene dominant primary clade is scored with a sum of all known sedative terpenes and the group median is 12.9%, a high level of secondary sedative terpenes, with some samples having sedative terpenes at over 20%.

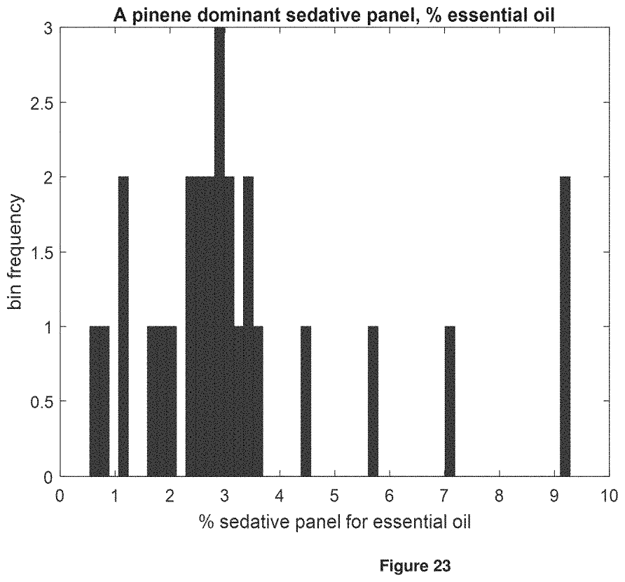

As shown in FIG. 23, the corresponding same sedative scoring for the alpha pinene dominant primary clade leads to a median of 2.8%, with a high of 9%. Therefore, the alpha pinene clade is a less sedative clade, but within the clade are a few that have a mild secondary terpene sedative scoring.

The results demonstrate that a multi-tier classification system can be used to efficiently classify plant strains, first by constructing familial clades based on grouping according to the dominant terpenes in each strain. Within each clade, the secondary terpenes of the chemovars can then be assessed according to one or more properties such as ancestry, agricultural need and therapeutic activity.

### Example 2: Examples of Certain Non-Limiting Embodiments

Listed hereafter are non-limiting examples of certain embodiments of the technology.

A1. A method of classifying a plurality of strains of a plant according to chemotype, comprising:
(a) obtaining a sample from each of the plurality of strains;
(b) for each sample, obtaining a measured amount of one or more individual analytes in the sample, and a measured amount of the total analytes in the sample, wherein the analytes belong to the same chemical class;
(c) for each plant sample, based on the measured amounts in (b):
(i) determining the abundance of the one or more individual analytes in the sample relative to the total amount of analytes in the sample, thereby obtaining the relative abundance of the one or more individual analytes in the sample,
(ii) determining the order of relative abundance, from highest to lowest relative abundance or from lowest to highest relative abundance, of the one or more individual analytes in the sample, and
(iii) based on (i) and (ii), determining an abundance profile of the analytes for each plant sample;
(d) optionally, for each plant sample, determining whether the sample is an outlier and, if the plant sample is an outlier, not subjecting the sample to (e) and (f) or,
determining the difference between the original analyte abundance profile of the sample and the analyte abundance profile that renders the sample an outlier and, based on the difference, reconstructing the original analyte profile of the sample before subjecting the sample to (e) and (f);
(e) for each plant sample not identified as an outlier or, if identified as an outlier, reconstructed to its original abundance profile, normalizing the measured amounts of the one or more individual analytes, thereby obtaining, for each plant sample, a normalized abundance profile comprising normalized analyte levels of the one or more individual analytes; and
(f) based on the normalized abundance profiles of the analytes for each plant sample, assigning plant samples comprising the same normalized abundance profiles to a group, wherein each group is a primary clade that comprises plant samples comprising the same chemotype.

A2. The method of embodiment A1, further comprising identifying one or more secondary clades in at least one primary clade, the method comprising:
(1) for each plant sample in at least one primary clade, obtaining the identity and/or normalized measured amount of (i) one or more additional analytes, or (ii) a mixture of one or more individual analytes in (a) and one or more additional analytes, wherein the additional analytes are associated with heredity and/or a known therapeutic effect and wherein the additional analytes are different than the individual analytes in (a);
(2) for each plant sample, based on the identity and/or normalized measured amount of amount of (i) or (ii), obtaining one or more profiles selected from among a heredity profile of analytes and a therapeutic profile of the analytes of (i) or (ii); and
(3) identifying plant samples within each primary clade that comprise the same heredity profiles and/or therapeutic profiles, as belonging to the same secondary clade.

A3. The method of embodiment A1 or A2, wherein determining whether the sample is an outlier comprises:
(a) identifying whether the total amount of the analyte in the sample is less than a threshold amount and, if the amount is less than the threshold amount, identifying the sample as an outlier; and/or
(b) comparing the measured amount of at least one individual first analyte to a reference amount of the first analyte, and/or comparing the ratio of the measured amounts of at least one individual first analyte and at least one individual second analyte to a reference ratio of the amounts of the first analyte and the second analyte, and if the measured amount and/or ratio is different than the reference amount or ratio, identifying the plant sample as an outlier.

A4. The method of any one of embodiments A1 to A3, wherein in (f), assigning plant samples comprising the same normalized abundance profiles to a group comprises:
performing a clustering analysis to obtain one or more clusters, wherein each cluster is assigned an average abundance profile;
representing the average abundance profile as a centroid vector;
representing the normalized abundance profile of each plant sample as a vector;
identifying all plant samples whose normalized abundance profile vector distances to the centroid vector are at or below a minimum value as having the same abundance profiles and belonging to the same cluster; and
identifying each cluster comprising a unique centroid vector that is different than the centroid vectors of all the other clusters obtained by the clustering analysis as a primary clade.

A5. The method of any one of embodiments A2 to A4, wherein in (3), identifying plant samples within each primary clade that comprise the same heredity profiles and/or therapeutic profiles comprises:
performing a clustering analysis to obtain one or more clusters, wherein each cluster is assigned an heredity profile or an average therapeutic profile;
representing the average heredity profile or the average therapeutic profile as a centroid vector;
representing the heredity profile or therapeutic profile of each plant sample as a vector;
identifying all plant samples whose heredity profile vector or therapeutic profile vector distances to the centroid vector are at or below a minimum value as having the same heredity profiles or therapeutic profiles and belonging to the same cluster; and
identifying each cluster comprising a unique centroid vector that is different than the centroid vectors of all the other clusters obtained by the clustering analysis as a secondary clade.

A6. The method of any one of embodiments A2 to A5 wherein, for (1), if the identity and/or normalized measured amount of a mixture of one or more individual analytes in (a) and one or more additional analytes is used, the one or more individual analytes in (a) are modified by a weighting factor.

A7. The method of embodiment A6, wherein at least one secondary clade comprises two or more plant strains comprising the same therapeutic profile and the weighting factor is based on potency.

A8. The method of any one of embodiments A1 to A7, wherein for (b) (iii) (e), a subset of the one or more individual analytes is selected for normalizing the measured amounts of the one or more individual analytes.

A9. The method of embodiment A8, wherein the subset comprises individual analytes comprising 3%, 4%, 5%, 6%, 7%, 8%, 9%, 10%, 11%, 12%, 13%, 14%, 15%, 16%, 17%, 18%, 19%, 20% or more by weight of the total amount by weight of the total amount of all the analytes recovered from the plant sample.

A10. The method of embodiment A1, wherein the analytes are terpenes.

A10.1. The method of any one of embodiments A2 to A9, wherein the analytes are terpenes

A11. The method of any one of embodiments A1 to A10.1, wherein the plant strains are Cannabis strains.

A12. The method of any one of embodiments A10, A10.1 or A11, wherein for (e), a subset of the one or more individual terpenes is selected for normalizing the measured amounts of the one or more individual terpenes.

A13. The method of embodiment A12, wherein the subset of terpenes comprises beta myrcene, beta caryophyllene, limonene, alpha pinene, beta farnesene, and terpinolene.

A14. The method of embodiment A13, wherein the subset of terpenes further comprises humulene, beta pinene, and alpha farnesene.

A15. The method of any one of embodiments A11 to A14, wherein determining whether the sample is an outlier further comprises measuring the ratio of tetrahydrocannabinol (THC) to tetraydrocannabinolic acid (THCA) and, if the ratio is at or above a threshold value, identifying the sample as an outlier.

A16. The method of embodiment A15, wherein the ratio is at or above 1:10.

A17. The method of any one of embodiments A10 to A16, comprising performing part (d) and wherein determining whether the sample is an outlier comprises one or more of:
1) if the ratio of beta caryophyllene:humulene is not between 2:1 to 6:1, identifying the sample as an outlier;
2) if the amount of alpha pinene is greater than two times the limit of quantitation (LOQ), beta pinene must be detected or the sample is identified as an outlier;
3) if beta pinene is at limit of quantitation (LOQ), alpha pinene must be detected or the sample is identified as an outlier;
4) if the ratio of alpha pinene:beta pinene is not between 0.3:1 to 6:1, identifying the sample as an outlier;
5) if the ratio of terpinolene:3-carene is not between 10:1 to 38:1, identifying the sample as an outlier;
6) if the ratio of terpinolene:alpha phellandrene is not between 5:1 to 30:1, identifying the sample as an outlier;
7) if the ratio of terpinolene:alpha pinene is not between 20:1 to 100:1, identifying the sample as an outlier;
8) if the ratio of alpha terpineol:fenchol is not between 0.3:1 to 2.5:1, identifying the sample as an outlier;
9) if the ratio of terpinolene:gamma terpinene ratios is not between 20:1 to 120:1, identifying the sample as an outlier;
10) if the sample comprises about or less than about 0.7, 0.75, 0.8, 0.85, 0.9, 0.95 or 1% total terpenes by weight, based on the total dry weight of the sample, identifying the sample as an outlier; and
11) if the THC content of the sample is 10% or more of the THCA content, identifying the sample as an outlier.

A18. The method of embodiment A17, wherein if the sample comprises about or less than about 0.9% total terpenes by weight, based on the total dry weight of the sample, the sample is identified as an outlier.

A19. The method of any one of embodiments A10 to A18, comprising, in (d), determining the difference between the original terpene abundance profile of the sample and the terpene abundance profile that renders the sample an outlier and, based on the difference, reconstructing the original terpene profile of the sample before subjecting the sample to (e) and (f).

A20. The method of embodiment A19, wherein determining the difference between the original terpene abundance profile of the sample and the terpene abundance profile that renders the sample an outlier comprises determining the decay profile of one or more terpenes in the sample, determining the storage time of the sample, identifying and/or quantitating terpene degradation products in the sample and/or determinating the estimated dissipation of one or more terpenes in the sample.

A21. The method of any one of embodiments A2 to A20 wherein one or more additional analytes for identifying secondary clades has a low volatilization rate.

A22. The method of embodiment A21, wherein the one or more additional analytes is/are terpene(s).

A23. The method of embodiment A22, wherein the one or more terpenes are selected from among monoterpene alcohols, sesquiterpenes, sesquiterpene alcohols or combinations thereof.

A24. The method of embodiments A22 or A23, wherein the one or more terpenes are selected from among alpha bisabolol, alpha terpineol, guiaol, nerolidol, fenchol and linalool.

A25. The method of any one of embodiments A2 to A9 and A10.1 to A25, wherein at least one secondary clade is obtained based on scoring one or more of the analytes for heredity, thereby obtaining at least one secondary clade wherein the plant strains that are members of the clade share the same average heredity profile.

A25.1. The method of any one of embodiments A10.1 to A24, wherein at least one secondary clade is obtained based on scoring one or more of the terpenes for heredity, thereby obtaining at least one secondary clade wherein the plant strains that are members of the clade share the same average heredity profile.

A26. The method of embodiment A25.1, wherein the terpenes that are scored for heredity comprise one or more terpenes selected from among monoterpene alcohols, sesquiterpenes, sesquiterpene alcohols or combinations thereof.

A27. The method of embodiment A25.1 or A26, wherein the terpenes that are scored for heredity comprise one or more terpenes selected from among alpha bisabolol, alpha terpineol, guiaol, nerolidol, fenchol and linalool.

A28. The method of any one of embodiments A25 to A27, wherein the average heredity profile is further correlated with therapeutic activity, thereby obtaining an average therapeutic profile for the secondary clade.

A29. The method of any one of embodiments A2 to A9 and A10.1 to A28, wherein at least one secondary clade is obtained based on scoring one or more of the analytes for one or more therapeutic effects, thereby obtaining at least one secondary clade wherein the plant strains that are members of the clade share the same average therapeutic profile.

A29.1. The method of any one of embodiments A10.1 to A28, wherein at least one secondary clade is obtained based on scoring one or more of the terpenes for one or more therapeutic effects, thereby obtaining at least one secondary clade wherein the plant strains that are members of the clade share the same average therapeutic profile.

A30. The method of embodiment A29 or A29.1, wherein the therapeutic effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

A31. The method of embodiment A30, wherein at least one therapeutic effect is AChEI.

A32. The method of embodiment A31, wherein the analytes are terpenes and the terpenes that are scored comprise one or more terpenes selected from among alpha pinene, eucalyptol, 3 carene, alpha terpinene, gamma terpinene, cis ocimene, trans ocimene and beta caryophyllene oxide.

A33. The method of any one of embodiments A30 to A32, wherein at least one therapeutic effect is analgesic.

A34. The method of embodiment A33, wherein the analytes are terpenes and the terpenes that are scored comprise one or more terpenes selected from among alpha bisabolol, alpha terpineol, alpha phellandrene and nerolidol.

A35. The method of embodiment A29.1, wherein the therapeutic effect is on the brain waves.

A36. The method of embodiment A35, wherein the therapeutic effect is gender selective.

A37. The method of embodiment A35 or A36, wherein the terpenes that are scored comprise one or more terpenes selected from terpinolene, (+) limonene, (+) alpha pinene and (+) beta pinene.

A38. The method of any one of embodiments A1 to A37, wherein in (b), the number of individual analytes whose amounts are measured is between about 5 individual analytes to about 45, 50, 55, 60, 65, 70, 75, 80, 85, 90, 95, 100 or more individual analytes.

A39. The method of embodiment A38, wherein the analytes are terpenes.

A40. The method of embodiment A39, wherein the number of terpenes whose amounts are measured in (b) is between about 10 terpenes to about 45, 50, 55, 60, 65, 70, 75, 80, 85, 90, 95, 100 or more terpenes.

A41. The method of embodiment A39, wherein the number of terpenes whose amounts are measured in (b) is between about 20 terpenes to about 45, 50, 55, 60, 65 or 70 terpenes.

A41.1. The method of embodiment A40 or A41, wherein the terpenes comprise one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, O-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin

A42. The method of embodiment A41 or A41.1, wherein the number of terpenes whose amounts are measured in (b) is 43.

A43. The method of any one of embodiments A40 to A42, wherein the number of terpenes subjected to (c) (iii) through (f) and (1) through (3) to obtain primary and/or secondary clades is a subset of the number of terpenes whose amounts are measured in (b).

A44. The method of embodiment A43, wherein the number of terpenes in the subset is 10, 11, 12, 13, 14, 15, 16, 17, 18, 19 or 20 or more terpenes.

A45. The method of embodiment A44, wherein the number of terpenes in the subset is 20.

A46. The method of embodiment A44, wherein the number of terpenes in the subset is 17.

A47. The method of any one of embodiments A43 to A46, wherein the number of terpenes subjected to (c) (iii) through (f) to obtain primary clades is at least 3, 4, 5, 6, 7, 8, 9, 10, 11 or 12 terpenes.

A48. The method of embodiment A47, wherein the number of terpenes subjected to (c) (iii) through (f) to obtain primary clades is at least 6 terpenes.

A49. The method of embodiment A48, wherein the number of terpenes subjected to (c) (iii) through (f) to obtain primary clades is 6 terpenes.

A50. The method of embodiments A47 or A48, wherein the number of terpenes subjected to (c) (iii) through (f) to obtain primary clades is at least 9 terpenes.

A51. The method of embodiment A50, wherein the number of terpenes subjected to (c) (iii) through (f) to obtain primary clades is 9 terpenes.

A52. The method of any one of embodiments A48 to A51, wherein at least one of the terpenes is beta farnesene.

A53. The method of embodiments A48 or A49, wherein the 6 terpenes are beta myrcene, beta caryophyllene, limonene, alpha pinene, beta farnesene and terpinolene.

A54. The method of embodiments A50 or A51, wherein the 9 terpenes are beta myrcene, beta caryophyllene, limonene, alpha pinene, beta farnesene, terpinolene, humulene, beta pinene, alpha farnesene.

A55. The method of any of embodiments A1 to A54, further comprising obtaining a classification system, wherein:
the classification system comprises one or more primary clades obtained according to (f); or
the classification system comprises one or more primary clades obtained according to (f) and comprises one or more secondary clades obtained according to (3).

A56. The method of any one of embodiments A1 to A55, wherein the number of primary clades is 3, 4, 5, 6, 7, 8, 9, 10, 11 or 12.

A57. The method of embodiment A56, wherein the number of primary clades is 7.

B1. A classification system obtained by the method of any one of embodiments A55 to A57.

C1. A classification system, comprising:
(a) a first classification tier comprising one or more primary clades, wherein the one or more of primary clades all comprise one or more strains of plants belonging to the same genus and wherein each primary clade comprises one or more strains of plants belonging to the same genus that share a unique abundance profile of analytes that is different than the abundance profiles of analytes of the strains of plants in the other primary clades; and
(b) a second classification tier, comprising one or more secondary clades, wherein:
the plant strains or a subset thereof in at least one primary clade are grouped into one or more secondary clades, wherein each secondary clade comprises one or more strains of plants that share at least one unique profile selected from among (i) a unique heredity profile of analytes, and/or (iii) a unique therapeutic profile of analytes, wherein the shared unique profile/profiles of the plants in each secondary clade are different than the corresponding profiles of the plants in the other secondary clades,
the profiles in the second classification tier comprise analytes that are different than the analytes of the profiles in the first classification tier, or the profiles in the second classification tier comprise analytes that are a mixture of one or more analytes of the profiles in the first classification tier and one or more analytes that are different than the analytes of the profiles in the first classification tier, and
the analytes in the first classification tier and the analytes in the second classification tier belong to the same chemical class.

C2. The system of embodiment C1, wherein the analytes are terpenes.

C3. The system of embodiments C1 or C2, wherein the plant strains are Cannabis strains.

C4. The system of embodiments C2 or C3, wherein the terpenes comprise one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, O-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol. Hedycaryol, γ-eudesmol. Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin

C5. The system of any one of embodiments C2 to C4, wherein the abundance profiles are obtained based on the abundances of at least 5, 6, 7, 8, 9, 10, 11 or 12 terpenes in each plant strain.

C6. The system of embodiment C5, wherein the abundance profiles are obtained based on the abundances of at least 6 terpenes.

C7. The system of embodiment C5, wherein the abundance profiles are obtained based on the abundances of 6 terpenes.

C8. The system of embodiments C5 or C6, wherein the abundance profiles are obtained based on the abundances of at least 9 terpenes.

C9. The system of embodiment C8, wherein the abundance profiles are obtained based on the abundances of 9 terpenes.

C10. The system of any one of embodiments C5 to C9, wherein at least one of the terpenes is beta farnesene.

C11. The system of embodiments C6 or C7, wherein the 6 terpenes are beta myrcene, beta caryophyllene, limonene, alpha pinene, beta farnesene and terpinolene.

C12. The system of embodiments 08 or 09, wherein the 9 terpenes are beta myrcene, beta caryophyliene, limonene, alpha pinene, beta farnesene, terpinolene, humulene, beta pinene and alpha farnesene.

C13. The system of any one of embodiments C2 to C12, wherein the total number of abundance, heredity and/or therapeutic profiles are obtained based on the abundance, heredity scoring and/or therapeutic scoring of 10, 11, 12, 13, 14, 15, 16, 17, 18, 19 or 20 or more terpenes.

C14. The system of embodiment C13, wherein the total number of abundance, heredity and/or therapeutic profiles are obtained based on the abundance, heredity scoring and/or therapeutic scoring of 20 terpenes.

C15. The system of embodiment C13, wherein the total number of abundance, heredity and/or therapeutic profiles are obtained based on the abundance, heredity scoring and/or therapeutic scoring of 17 terpenes.

C16. The system of any one of embodiments C2 to C15, wherein at least one secondary clade is obtained based on scoring one or more of the terpenes for heredity, wherein the plant strains that are members of the clade share the same average heredity profile.

C17. The system of embodiment C16, wherein the terpenes that are scored for heredity comprise one or more terpenes selected from among monoterpene alcohols, sesquiterpenes, sesquiterpene alcohols or combinations thereof.

C18. The system of embodiment C16 or C17, wherein the terpenes that are scored for heredity comprise one or more terpenes selected from among alpha bisabolol, alpha terpineol, guiaol, nerolidol, fenchol and linalool.

C19. The system of any one of embodiments C16 to C18, wherein the average heredity profile is further correlated with therapeutic activity and the secondary clade comprises an average heredity profile and an average therapeutic profile.

C20. The system of any one of embodiments C2 to C19, wherein at least one secondary clade is obtained based on scoring one or more of the terpenes for one or more therapeutic effects, wherein the plant strains that are members of the clade share the same average therapeutic profile.

C21. The system of embodiments C19 or C20, wherein the therapeutic effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

C22. The system of embodiment C21, wherein at least one therapeutic effect is AChEI.

C23. The system of embodiment C22, wherein the terpenes that are scored comprise one or more terpenes selected from among alpha pinene, eucalyptol, 3 carene, alpha terpinene, gamma terpinene, cis ocimene, trans ocimene and beta caryophyllene oxide.

C24. The system of any one of embodiments C20 to C23, wherein at least one therapeutic effect is analgesic.

C25. The system of embodiment C24, wherein the terpenes that are scored comprise one or more terpenes selected from among alpha bisabolol, alpha terpineol, alpha phellandrene and nerolidol.

C26. The system of embodiment C20, wherein the therapeutic effect is on the brain waves.

C27. The system of embodiment C26, wherein the therapeutic effect is gender selective.

C28. The system of embodiments C26 or C27, wherein the terpenes that are scored comprise one or more terpenes selected from terpinolene, (+) limonene, (+) alpha pinene and (+) beta pinene.

C29. The system of any one of embodiments C1 to C28, wherein the number of primary clades is 3, 4, 5, 6, 7, 8, 9, 10, 11 or 12.

C30. The system of embodiment C29, wherein the number of primary clades is 7.

D1. A method of classifying a plant test sample, comprising:
(a) obtaining a measured amount of one or more individual analytes in the test sample;
(b) optionally, (i) comparing the measured amount of at least one individual first analyte to a reference amount of the first analyte, and/or (ii) comparing the ratio of the measured amounts of at least one individual first analyte and at least one individual second analyte to a reference ratio of the amounts of the first analyte and the second analyte, and if the measured amount and/or ratio is different than the reference amount or ratio, identifying the plant sample as an outlier and excluding the plant sample from the classification system;
(c) normalizing the measured amount of each of the one or more individual analytes, thereby providing normalized individual analyte levels;
(d) obtaining an abundance profile of analytes for the test sample, wherein the abundance profile comprises the normalized individual analyte levels;
(e) comparing the abundance profile of analytes of the test sample to the average central value of the abundance profile of analytes of each primary clade of the classification system of any one of embodiments B1 and C1 to C30, thereby providing a comparison; and
(f) based on the comparison, assigning the test sample to a primary clade selected from among the plurality of primary clades, thereby classifying the test sample.

D2. The method of embodiment D1, further comprising:
(1) obtaining, for the plant test sample, the identity and/or normalized measured amount of (i) one or more additional analytes, or (ii) a mixture of one or more individual analytes in (a) and one or more additional analytes, wherein the additional analytes are associated with heredity and/or a known therapeutic effect and wherein the additional analytes are different than the individual analytes in (a);
(2) obtaining one or more profiles selected from among a heredity profile, a therapeutic profile and an abundance profile based on the identity and/or measured amount of (i) or (ii); and
(3) comparing each of the one or more profiles of the test sample from (2) to the average central value of a corresponding profile of each secondary clade of the plant classification system of any one of embodiments B1 and C1 to C30, thereby providing a comparison; and
(d) based on the comparison, assigning the test sample to a secondary clade selected from among the plurality of secondary clades, thereby classifying the test sample.

D3. The method of embodiments D1 or D2, wherein the comparison is by Euclidean analysis.

D4. The method of any one of embodiments D1 to D3, wherein the analytes are terpenes.

D5. The method of any one of embodiments D1 to D4, wherein the test sample is from a Cannabis plant strain.

E1. A method of breeding one or more plant strains, comprising:
(i) obtaining a plurality of plant strains or samples therefrom;
(ii) classifying the plurality of plant strains according to the method of any one of embodiments A1 to A57;
(iii) based on the classification, identifying one or more plant strains belonging to a primary clade of interest and, optionally, a secondary clade of interest; and
(iv) breeding the one or more plant strains identified according to (iii).

E2. The method of embodiment E1, wherein the identification in (iii) is of an analyte abundance profile of interest in a primary clade.

E3. The method of embodiment E2, wherein the analyte abundance profile is one that confers resistance to growth of the one or more plant strains in an environmental condition or a geographic location.

E4. The method of embodiment E2, wherein the analyte abundance profile is one that is favorable for growth of the one or more plant strains in an environmental condition or a geographic location.

E5. The method of any one of embodiments E1 to E4, wherein in (iii), one or more plant strains are identified as belonging to a primary clade of interest and at least one secondary clade of interest.

E6. The method of embodiment E5, wherein the identification of the at least one secondary clade of interest in (iii) is of a heredity profile.

E7. The method of embodiment E5, wherein the identification of the at least one secondary clade of interest in (iii) is of a therapeutic profile.

E8. The method of embodiment E7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

E9. The method of any one of embodiments E5 to E8, wherein in (iii), one or more plant strains are identified as belonging to a primary clade of interest and to more than one secondary clade of interest.

E10. The method of any one of embodiments E1 to E9, wherein the analytes are terpenes.

E11. The method of any one of embodiments E1 to E1 0, wherein the one or more plant strains are Cannabis strains.

F1. A method of cultivating one or more plant strains as a crop, comprising:
(i) obtaining a plurality of plant strains or samples therefrom;
(ii) classifying the plurality of plant strains according to the method of any one of embodiments A1 to A57;
(iii) based on the classification, identifying one or more plant strains belonging to a primary clade of interest and, optionally, a secondary clade of interest; and
(iv) cultivating the one or more plant strains identified according to (iii) as a crop.

F2. The method of embodiment F1, wherein the identification in (iii) is of an analyte abundance profile of interest in a primary clade.

F3. The method of embodiment F2, wherein the analyte abundance profile is one that confers resistance to growth of the one or more plant strains an environmental condition or a geographic location.

F4. The method of embodiment F2, wherein the analyte abundance profile is one that is favorable for growth of the one or more plant strains in an environmental condition or a geographic location.

F5. The method of any one of embodiments F1 to F4, wherein in (iii), one or more plant strains are identified as belonging to a primary clade of interest and at least one secondary clade of interest.

F6. The method of embodiment F5, wherein the identification of the at least one secondary clade of interest in (iii) is of a heredity profile.

F7. The method of embodiment F5, wherein the identification of the at least one secondary clade of interest in (iii) is of a therapeutic profile.

F8. The method of embodiment F7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

F9. The method of any one of embodiments F5 to F8, wherein in (iii), one or more plant strains are identified as belonging to a primary clade of interest and more than one secondary clade of interest.

F10. The method of any one of embodiments F1 to F9, wherein the analytes are terpenes.

F11. The method of any one of embodiments F1 to F10, wherein the one or more plant strains are Cannabis strains.

G1. A method of treating a subject with one or more plant strains or a portion thereof or an extract thereof, comprising:
(i) obtaining a plurality of plant strains or samples therefrom;
(ii) classifying the plurality of plant strains according to the method of any one of embodiments A1 to A57;
(iii) based on the classification, identifying one or more plant strains belonging to a primary clade of interest and at least one secondary clade of interest based on a therapeutic profile of the analytes of the plant strains; and
(iv) treating the subject with the one or more plant strains identified according to (iii), or with a portion thereof, or with an extract thereof.

G2. The method of embodiment G1, wherein the subject is a human or an animal.

G3. The method of embodiments G1 or G2, wherein the portion thereof is a seed, flower, stem or leaf of the one or more plant strains.

G4. The method of any one of embodiments G1 to G3, wherein the subject is treated with a portion or an extract of the one or more plant strains.

G5. The method of any one of embodiments G1 to G4, wherein the treatment is administered orally, topically, or through inhalation.

G6. The method of any one of embodiments G1 to G5, wherein the treatment is self-administered, or is administered by an entity other than the subject.

G7. The method of any one of embodiments G1 to G6, wherein the identification in (iii) comprises identification of an analyte abundance profile of interest in the primary clade.

G8. The method of any one of embodiments G1 to G7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

G9. The method of any one of embodiments G1 to G8, wherein in (iii), one or more plant strains are identified as belonging to a primary clade of interest and to more than one secondary clade of interest.

G10. The method of any one of embodiments G1 to G9, wherein the analytes are terpenes.

G11. The method of any one of embodiments G1 to G10, wherein the one or more plant strains are Cannabis strains.

H1. A method of breeding a plant strain, comprising:
(i) obtaining a plant strain or a sample therefrom;
(ii) classifying the plant strain by the method of any one of embodiments D1 to D5;
(iii) based on the classification, identifying the plant strain as belonging to a primary clade of interest and, optionally, a secondary clade of interest; and
(iv) breeding the plant strain identified according to (iii).

H2. The method of embodiment H1, wherein the identification in (iii) is of an analyte abundance profile of interest in a primary clade.

H3. The method of embodiment H2, wherein the analyte abundance profile is one that confers resistance to growth of the plant strains in an environmental condition or a geographic location.

H4. The method of embodiment H2, wherein the analyte abundance profile is one that is favorable for growth of the plant strains in an environmental condition or a geographic location.

H5. The method of any one of embodiments H1 to H4, wherein in (iii), one or plant strains are identified as belonging to a primary clade of interest and at least one secondary clade of interest.

H6. The method of embodiment H5, wherein the identification of the at least one secondary clade of interest in (iii) is of a heredity profile.

H7. The method of embodiment H5, wherein the identification of the at least one secondary clade of interest in (iii) is of a therapeutic profile.

H8. The method of embodiment H7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

H9. The method of any one of embodiments H5 to H8, wherein in (iii), the plant strain is identified as belonging to a primary clade of interest and to more than one secondary clade of interest.

H10. The method of any one of embodiments H1 to H9, wherein the analytes are terpenes.

H11. The method of any one of embodiments H1 to H10, wherein the plant strain is a Cannabis strain.

I1. A method of cultivating a plant strain as a crop, comprising:
(i) obtaining a plant strain or a sample therefrom;
(ii) classifying the plant strain by the method of any one of embodiments D1 to D5;
(iii) based on the classification, identifying the plant strain as belonging to a primary clade of interest and, optionally, a secondary clade of interest; and
(iv) cultivating the plant strain identified according to (iii) as a crop.

I2. The method of embodiment I1, wherein the identification in (iii) is of an analyte abundance profile of interest in a primary clade.

I3. The method of embodiment I2, wherein the analyte abundance profile is one that confers resistance to growth of the plant strains in an environmental condition or a geographic location.

I4. The method of embodiment I2, wherein the analyte abundance profile is one that is favorable for growth of the plant strains in an environmental condition or a geographic location.

I5. The method of any one of embodiments I1 to I4, wherein in (iii), one or plant strains are identified as belonging to a primary clade of interest and at least one secondary clade of interest.

I6. The method of embodiment I5, wherein the identification of the at least one secondary clade of interest in (iii) is of a heredity profile.

I7. The method of embodiment I5, wherein the identification of the at least one secondary clade of interest in (iii) is of a therapeutic profile.

I8. The method of embodiment I7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

I9. The method of any one of embodiments I5 to I8, wherein in (iii), the plant strain is identified as belonging to a primary clade of interest and to more than one secondary clade of interest.

I10. The method of any one of embodiments I1 to I9, wherein the analytes are terpenes.

I11. The method of any one of embodiments I1 to I10, wherein the plant strain is a Cannabis strain.

J1. A method of treating a subject with a plant strain or a portion thereof or an extract thereof, comprising:
(i) obtaining a plant strain or a sample therefrom;
(ii) classifying the plant strain by the method of any one of embodiments D1 to D5;
(iii) based on the classification, identifying the plant strain as belonging to a primary clade of interest and at least one secondary clade of interest based on a therapeutic profile of the analytes of the plant strain; and
(iv) treating the subject with the plant strain identified according to (iii), or with a portion thereof, or with an extract thereof.

J2. The method of embodiment J1, wherein the subject is a human or an animal.

J3. The method of embodiments J1 or J2, wherein the portion thereof is a seed, flower, stem or leaf of the plant strain.

J4. The method of any one of embodiments J1 to J3, wherein the subject is treated with a portion or an extract of the plant strain.

J5. The method of any one of embodiments J1 to J4, wherein the treatment is administered orally, topically, or through inhalation.

J6. The method of any one of embodiments J1 to J5, wherein the treatment is self-administered, or the treatment is administered by an entity other than the subject.

J7. The method of any one of embodiments J1 to J6, wherein the identification in (iii) comprises identification of an analyte abundance profile of interest in the primary clade.

J8. The method of any one of embodiments J1 to J7, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

J9. The method of any one of embodiments J1 to J8, wherein in (iii), the plant strain is identified as belonging to a primary clade of interest and to more than one secondary clade of interest.

J10. The method of any one of embodiments J1 to J9, wherein the analytes are terpenes.

J11. The method of any one of embodiments J1 to J10, wherein the plant strain is a Cannabis strain.

K1. The method of any one of embodiments A1 to A57, D1-D5, E1-E1l, F1-F1 1, G1-G11, H1-H11, I1-I11 and J1-J11, wherein one or more of (c) to (f) of A1 are performed by a machine comprising one or more microprocessors and memory, wherein:
the memory comprises instructions for performing one or more of (c) to (f); and
the one or more microprocessors execute the instructions.

K2. The method of embodiment K1, wherein the machine comprising one or more microprocessors and memory further performs one or more of (1) to (3) of A2, wherein:
the memory comprises instructions for performing one or more of (1) to (3); and
the one or more microprocessors execute the instructions.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions that have been employed are used as terms of description and not of limitation and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method of classifying a plurality of strains of a plant according to chemotype, comprising:
(a) obtaining a sample from each of the plurality of strains;
(b) for each sample, obtaining a measured amount of one or more individual analytes in the sample, and a measured amount of the total analytes in the sample, wherein the analytes belong to the same chemical class;
(c) for each plant sample, based on the measured amounts in (b):
(i) determining the abundance of the one or more individual analytes in the sample relative to the total amount of analytes in the sample, thereby obtaining the relative abundance of the one or more individual analytes in the sample,
(ii) determining the order of relative abundance, from highest to lowest relative abundance or from lowest to highest relative abundance, of the one or more individual analytes in the sample, and
(iii) based on (i) and (ii), determining an abundance profile of the analytes for each plant sample;
(d) optionally, for each plant sample, determining whether the sample is an outlier and, if the plant sample is an outlier, not subjecting the sample to (e) and (f) or, determining the difference between the original analyte abundance profile of the sample and the analyte abundance profile that renders the sample an outlier and, based on the difference, reconstructing the original analyte profile of the sample before subjecting the sample to (e) and (f);
(e) for each plant sample not identified as an outlier or, if identified as an outlier, reconstructed to its original abundance profile, normalizing the measured amounts of the one or more individual analytes, thereby obtaining, for each plant sample, a normalized abundance profile comprising normalized analyte levels of the one or more individual analytes; and
(f) based on the normalized abundance profiles of the analytes for each plant sample, assigning plant samples comprising the same normalized abundance profiles to a group, wherein each group is a primary clade that comprises plant samples comprising the same chemotype.

2. The method of claim 1, further comprising identifying one or more secondary clades in at least one primary clade, the method comprising:
(1) for each plant sample in at least one primary clade, obtaining the identity and/or normalized measured amount of (i) one or more additional analytes, or (ii) a mixture of one or more individual analytes in (a) and one or more additional analytes, wherein the additional analytes are associated with heredity and/or a known therapeutic effect and wherein the additional analytes are different than the individual analytes in (a);
(2) for each plant sample, based on the identity and/or normalized measured amount of amount of (i) or (ii), obtaining one or more profiles selected from among a heredity profile of analytes and a therapeutic profile of the analytes of (i) or (ii); and
(3) identifying plant samples within each primary clade that comprise the same heredity profiles and/or therapeutic profiles, as belonging to the same secondary clade.

3. The method of claim 1 or claim 2, wherein determining whether the sample is an outlier comprises:
(i) identifying whether the total amount of the analyte in the sample is less than a threshold amount and, if the amount is less than the threshold amount, identifying the sample as an outlier; and/or
(ii) comparing the measured amount of at least one individual first analyte to a reference amount of the first analyte, and/or comparing the ratio of the measured amounts of at least one individual first analyte and at least one individual second analyte to a reference ratio of the amounts of the first analyte and the second analyte, and if the measured amount and/or ratio is different than the reference amount or ratio, identifying the plant sample as an outlier.

4. The method of any one of claims 1-3, wherein in (f), assigning plant samples comprising the same normalized abundance profiles to a group comprises:
performing a clustering analysis to obtain one or more clusters, wherein each cluster is assigned an average abundance profile;
representing the average abundance profile as a centroid vector;
representing the normalized abundance profile of each plant sample as a vector;
identifying all plant samples whose normalized abundance profile vector distances to the centroid vector are at or below a minimum value as having the same abundance profiles and belonging to the same cluster; and
identifying each cluster comprising a unique centroid vector that is different than the centroid vectors of all the other clusters obtained by the clustering analysis as a primary clade.

5. The method of any one of claims 2-4, wherein in (3), identifying plant samples within each primary clade that comprise the same heredity profiles and/or therapeutic profiles comprises:
performing a clustering analysis to obtain one or more clusters, wherein each cluster is assigned an heredity profile or an average therapeutic profile;
representing the average heredity profile or the average therapeutic profile as a centroid vector;
representing the heredity profile or therapeutic profile of each plant sample as a vector;
identifying all plant samples whose heredity profile vector or therapeutic profile vector distances to the centroid vector are at or below a minimum value as having the same heredity profiles or therapeutic profiles and belonging to the same cluster; and
identifying each cluster comprising a unique centroid vector that is different than the centroid vectors of all the other clusters obtained by the clustering analysis as a secondary clade.

6. The method of any one of claims 2-5 wherein, for (1), if the identity and/or normalized measured amount of a mixture of one or more individual analytes in (a) and one or more additional analytes is used, the one or more individual analytes in (a) are modified by a weighting factor.

7. The method of claim 6, wherein at least one secondary clade comprises two or more plant strains comprising the same therapeutic profile and the weighting factor is based on potency.

8. The method of any one of claims 1-7, wherein for (b) (iii) (e), a subset of the one or more individual analytes is selected for normalizing the measured amounts of the one or more individual analytes.

9. The method of claim 8, wherein the subset comprises individual analytes comprising 3%, 4%, 5%, 6%, 7%, 8%, 9%, 10%, 11%, 12%, 13%, 14%, 15%, 16%, 17%, 18%, 19%, 20% or more by weight of the total amount by weight of the total amount of all the analytes recovered from the plant sample.

10. The method of any one of claims 1-9, wherein the analytes are terpenes.

11. The method of any one of claims 1-10, wherein the plant strains are Cannabis strains.

12. The method of claim 10 or claim 11, wherein for (e), a subset of the one or more individual terpenes is selected for normalizing the measured amounts of the one or more individual terpenes.

13. The method of claim 12, wherein the subset of terpenes comprises beta myrcene, beta caryophyllene, limonene, alpha pinene, beta farnesene, and terpinolene.

14. The method of claim 13, wherein the subset of terpenes further comprises humulene, beta pinene, and alpha farnesene.

15. The method of any one of claims 11-14, wherein determining whether the sample is an outlier further comprises measuring the ratio of tetrahydrocannabinol (THC) to tetraydrocannabinolic acid (THCA) and, if the ratio is at or above a threshold value, identifying the sample as an outlier.

16. The method of claim 15, wherein the ratio is at or above 1:10.

17. The method of any one of claims 10-16, comprising performing part (d) and wherein determining whether the sample is an outlier comprises one or more of:
1) if the ratio of beta caryophyllene:humulene is not between 2:1 to 6:1, identifying the sample as an outlier;
2) if the amount of alpha pinene is greater than two times the limit of quantitation (LOQ), beta pinene must be detected or the sample is identified as an outlier;
3) if beta pinene is at limit of quantitation (LOQ), alpha pinene must be detected or the sample is identified as an outlier;
4) if the ratio of alpha pinene:beta pinene is not between 0.3:1 to 6:1, identifying the sample as an outlier;
5) if the ratio of terpinolene:3-carene is not between 10:1 to 38:1, identifying the sample as an outlier;
6) if the ratio of terpinolene:alpha phellandrene is not between 5:1 to 30:1, identifying the sample as an outlier;
7) if the ratio of terpinolene:alpha pinene is not between 20:1 to 100:1, identifying the sample as an outlier;
8) if the ratio of alpha terpineol:fenchol is not between 0.3:1 to 2.5:1, identifying the sample as an outlier;
9) if the ratio of terpinolene:gamma terpinene ratios is not between 20:1 to 120:1, identifying the sample as an outlier;
10) if the sample comprises about or less than about 0.7, 0.75, 0.8, 0.85, 0.9, 0.95 or 1% total terpenes by weight, based on the total dry weight of the sample, identifying the sample as an outlier; and
11) if the THC content of the sample is 10% or more of the THCA content, identifying the sample as an outlier.

18. The method of any one of claims 10-17, comprising, in (d), determining the difference between the original terpene abundance profile of the sample and the terpene abundance profile that renders the sample an outlier and, based on the difference, reconstructing the original terpene profile of the sample before subjecting the sample to (e) and (f).

19. The method of claim 18, wherein determining the difference between the original terpene abundance profile of the sample and the terpene abundance profile that renders the sample an outlier comprises determining the decay profile of one or more terpenes in the sample, determining the storage time of the sample, identifying and/or quantitating terpene degradation products in the sample and/or determinating the estimated dissipation of one or more terpenes in the sample.

20. The method of any one of claims 10-19, wherein at least one secondary clade is obtained based on scoring one or more of the terpenes for heredity, thereby obtaining at least one secondary clade wherein the plant strains that are members of the clade share the same average heredity profile.

21. The method of claim 20, wherein the terpenes that are scored for heredity comprise one or more terpenes selected from among alpha bisabolol, alpha terpineol, guiaol, nerolidol, fenchol and linalool.

22. The method of any one of claims 10-21, wherein at least one secondary clade is obtained based on scoring one or more of the terpenes for one or more therapeutic effects, thereby obtaining at least one secondary clade wherein the plant strains that are members of the clade share the same average therapeutic profile.

23. The method of claim 22, wherein the therapeutic effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

24. The method of claim 22 or claim 23, wherein the terpenes that are scored comprise one or more terpenes selected from among alpha pinene, eucalyptol, 3 carene, alpha terpinene, gamma terpinene, cis ocimene, trans ocimene and beta caryophyllene oxide, alpha bisabolol, alpha terpineol, alpha phellandrene and nerolidol.

25. The method of claim 22, wherein the therapeutic effect is on the brain waves.

26. The method of claim 25, wherein the therapeutic effect is gender selective.

27. The method of claim 25 or claim 26, wherein the terpenes that are scored comprise one or more terpenes selected from terpinolene, (+) limonene, (+) alpha pinene and (+) beta pinene.

28. The method of any one of claims 1-27, wherein in (b), the number of individual analytes whose amounts are measured is between about 5 individual analytes to about 45, 50, 55, 60, 65, 70, 75, 80, 85, 90, 95, 100 or more individual analytes.

29. The method of claim 28, wherein the analytes are terpenes.

30. The method of claim 29, wherein the terpenes comprise one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

31. The method of claim 29 or claim 30, wherein the number of terpenes subjected to (c) (iii) through (f) and (1) through (3) to obtain primary and/or secondary clades is a subset of the number of terpenes whose amounts are measured in (b).

32. The method of any one of claims 1-31, further comprising obtaining a classification system, wherein:
the classification system comprises one or more primary clades obtained according to (f); or
the classification system comprises one or more primary clades obtained according to (f) and comprises one or more secondary clades obtained according to (3).

33. The classification system obtained by the method of claim 32.

34. A classification system, comprising:
(a) a first classification tier comprising one or more primary clades, wherein the one or more of primary clades all comprise one or more strains of plants belonging to the same genus and wherein each primary clade comprises one or more strains of plants belonging to the same genus that share a unique abundance profile of analytes that is different than the abundance profiles of analytes of the strains of plants in the other primary clades; and
(b) a second classification tier, comprising one or more secondary clades, wherein:
the plant strains or a subset thereof in at least one primary clade are grouped into one or more secondary clades, wherein each secondary clade comprises one or more strains of plants that share at least one unique profile selected from among (i) a unique heredity profile of analytes, and/or (iii) a unique therapeutic profile of analytes, wherein the shared unique profile/profiles of the plants in each secondary clade are different than the corresponding profiles of the plants in the other secondary clades,
the profiles in the second classification tier comprise analytes that are different than the analytes of the profiles in the first classification tier, or the profiles in the second classification tier comprise analytes that are a mixture of one or more analytes of the profiles in the first classification tier and one or more analytes that are different than the analytes of the profiles in the first classification tier, and
the analytes in the first classification tier and the analytes in the second classification tier belong to the same chemical class.

35. The system of claim 34, wherein the analytes are terpenes.

36. The system of claim 34 or claim 35, wherein the plant strains are Cannabis strains.

37. The system of claim 35 or claim 36, wherein the terpenes comprise one or more that are selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin

38. The system of any one of claims 35-37, wherein the abundance profiles are obtained based on the abundances of at least 5, 6, 7, 8, 9, 10, 11 or 12 terpenes in each plant strain.

39. The system of claim 38, wherein the abundance profiles are obtained based on the abundances of at least 6 terpenes.

40. The system of claim 39, wherein the 6 terpenes are beta myrcene, beta caryophyllene, limonene, alpha pinene, beta farnesene and terpinolene.

41. The system of any one of claims 35 to 40, wherein the total number of abundance, heredity and/or therapeutic profiles are obtained based on the abundance, heredity scoring and/or therapeutic scoring of 10, 11, 12, 13, 14, 15, 16, 17, 18, 19 or 20 or more terpenes.

42. The system of any one of claims 33 to 41, wherein the number of primary clades is 3, 4, 5, 6, 7, 8, 9, 10, 11 or 12.

43. A method of breeding one or more plant strains, or for cultivating one or more plant strains as a crop, comprising:
(i) obtaining a plurality of plant strains or samples therefrom;
(ii) classifying the plurality of plant strains according to the method of any one of claims 1-32;
(iii) based on the classification, identifying one or more plant strains belonging to a primary clade of interest and, optionally, a secondary clade of interest; and
(iv) breeding the one or more plant strains identified according to (iii), or cultivating the one or more plant strains identified according to (iii) as a crop.

44. A method of treating a subject with one or more plant strains or a portion thereof or an extract thereof, comprising:
(i) obtaining a plurality of plant strains or samples therefrom;
(ii) classifying the plurality of plant strains according to the method of any one of claims 1-32;
(iii) based on the classification, identifying one or more plant strains belonging to a primary clade of interest and at least one secondary clade of interest based on a therapeutic profile of the analytes of the plant strains; and
(iv) treating the subject with the one or more plant strains identified according to (iii), or with a portion thereof, or with an extract thereof.

45. The method of claim 44, wherein the subject is a human or an animal.

46. The method of claim 44 or claim 45, wherein the portion thereof is a seed, flower, stem or leaf of the one or more plant strains.

47. The method of any one of claims 44-46, wherein the treatment is administered orally, topically, or through inhalation.

48. The method of any one of claims 44-47, wherein the treatment is self-administered, or is administered by an entity other than the subject.

49. The method of any one of claims 44-48, wherein the therapeutic profile is obtained based on scoring for one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective, gastro-protective effects, brain wave activity and gender-selective therapeutic activity.

50. The method of any one of claims 44-49, wherein the analytes are terpenes.

51. The method of any one of claims 44-50, wherein the one or more plant strains are Cannabis strains.
